SEQUENCE LISTING (31 sequences)

SEQ ID NO: 1 - DNA, 2434 bp, Homo sapiens
  CDS (60)..(1157)
gaattcccaggagcgagagccgcctacctgcagccgccgcccacggcacggcagccaccatggcgctcctgctgtgcttcgtgctcctgtgcggagtagtggatttcgccagaagtttgagtatcactactcctgaagagatgattgaaaaagccaaaggggaaactgcctatctgccgtgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtatagagcctccatatgtctcatctgtgctctccgtgttcctttcctttttttgatatatgaaaacctattctggtctaaattgtgttactagcctcaaaatacatcaaaaaataagttaatcaggaactgtacggaatatatttttaaaaatttttgtttggttatatcgaaatagttacaggcactaaagttagtaaagaaaagtttaccatctgaaaaagctggattttctttaagaggttgattataaagttttctaaatttatcagtacctaagtaagatgtagcgctttgaatatgaaatcataggtgaagacatgggtgaacttacttgcataccaagttgatacttgaataaccatctgaaagtggtacttgatcatttttaccattatttttaggatgtgtatttcatttatttatggcccaccagtctcccccaaattagtacagaaatatccatgacaaaattacttacgtatgtttgtacttggttttacagctcctttgaaaactctgtgtttggaatatctctaaaaacatagaaaacactacagtggtttagaaattactaattttacttctaagtcattcataaaccttgtctatgaaatgacttcttaaatatttagttgatagactgctacaggtaatagggacttagcaagctcttttatatgctaaaggagcatctatcagattaagttagaacatttgctgtcagccacatattgagatgacactaggtgcaatagcagggatagattttgttggtgagtagtctcatgccttgagatctgtggtggtcttcaaaatggtggccagccagatcaaggatgtagtatctcatagttcccaggtgatatttttcttattagaaaaatattataactcatttgttgtttgacacttatagattgaaatttcctaatttattctaaattttaagtggttctttggttccagtgctttatgttgttgttgtttttggatggtgttacatattatatgttctagaaacatgtaatcctaaatttaccctcttgaatataatccctggatgatattttttatcataaatgcagaataatcaaatacattttaagcaagttaagtgtcctccatcaattctgtattccagacttgggaggatgtacagttgctgttgtgtgatcaaacatgtctctgtgtagttccagcaaatcaagctgagctttgaaaaagtttgtcttagttttgtgaaggtgatttattcttagaaaaaaaaaaaaaaaaaaaaa

SEQ ID NO: 2 - protein, 365 aa, Homo sapiens
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKAGLIAGAIIGTLLALALIGLIIFCCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 3 - DNA, 93 bp, Saccharomyces cerevisiae
  CDS (1)..(93)
atgaaacaaattgaagacaagattgaagaaattctatcaaaaatttatcacattgaaaacgaaattgccagaattaaaaaactaattggcgaa

SEQ ID NO: 4 - protein, 31 aa, Saccharomyces cerevisiae
MKQIEDKIEEILSKIYHIENEIARIKKLIGE

SEQ ID NO: 5 - protein, 9 aa, Homo sapiens
CDCRGDCFC

SEQ ID NO: 6 - DNA, 90 bp, Homo sapiens
  CDS (1)..(90)
ctgcgcaagctgcgtaagcggctcctccgcgatgccgatgacctgctgcgcaagctgcgtaagcggctcctccgcgatgccgatgacctg

SEQ ID NO: 7 - protein, 30 aa, Homo sapiens
LRKLRKRLLRDADDLLRKLRKRLLRDADDL

SEQ ID NO: 8 - DNA, 30 bp, Artificial Sequence
  primer binding site
gtaggatccgccaccatggcgctcctgctg

SEQ ID NO: 9 - DNA, 30 bp, Artificial Sequence
  primer binding site
gcgcgaattcctatactatagacccatcct

SEQ ID NO: 10 - DNA, 28 bp, Artificial Sequence
  primer binding site
actagctagcgccgccaccatggcgctc

SEQ ID NO: 11 - DNA, 56 bp, Artificial Sequence
  primer binding site
gtgatctagactagtgatgatggtgatggtgctcgagagctttatttgaaggaggg

SEQ ID NO: 12 - DNA, 63 bp, Artificial Sequence
  oligonucleotide with “linker domain”
tcgaaccatcagcctccgcatctgcttccgcccctggatccgcggccgccattgagggccgcc

SEQ ID NO: 13 - DNA, 63 bp, Artificial Sequence
  oligonucleotide with “linker domain”
tcgaggcggccctcaatggcggccgcggatccaggggcggaagcagatgcggaggctgatggt

SEQ ID NO: 14 - DNA, 33 bp, Artificial Sequence
  oligonucleotide for cloning cRGD sequence
ggcctgcgattgccgtggtgattgcttttgcgc

SEQ ID NO: 15 - DNA, 33 bp, Artificial Sequence
  oligonucleotide for cloning cRGD sequence
ggccgcgcaaaagcaatcaccacggcaatcgca

SEQ ID NO: 16 - DNA, 51 bp, Artificial Sequence
  oligonucleotide for cloning apoEsequence
ggccctgcgcaagctgcgtaagcggctcctccgcgatgccgatgacctggc

SEQ ID NO: 17 - DNA, 51 bp, Artificial Sequence
  oligonucleotide for cloning apoEsequence
ggccgccaggtcatcggcatcgcggaggagccgcttacgcagcttgcgcag

SEQ ID NO: 18 - DNA, 78 bp, Artificial Sequence
  oligonucleotide for cloning glycine/serine linker
tcgaaggaggaggaggaagtggaggaggaggaagtggaggaggaggaagtggatccgcggccgccattgagggccgcc

SEQ ID NO: 19 - DNA, 78 bp, Artificial Sequence
  oligonucleotide for cloning glycine/serine linker
tcgaggcggccctcaatggcggccgcggatccacttcctcctcctccacttcctcctcctccacttcctcctcctcct

SEQ ID NO: 20 - DNA, 99 bp, Artificial Sequence
  oligonucleotide for cloning isoleucine variant GCN4 leucine zipper trimerization domain
ggccatgaaacaaattgaagacaagattgaagaaattctatcaaaaatttatcacattgaaaacgaaattgccagaattaaaaaactaattggcgaagc

SEQ ID NO: 21 - DNA, 99 bp, Artificial Sequence
  oligonucleotide for cloning isoleucine variant GCN4 leucine zipper trimerization domain
ggccgcttcgccaattagttttttaattctggcaatttcgttttcaatgtgataaatttttgatagaatttcttcaatcttgtcttcaatttgtttcat

SEQ ID NO: 22 - DNA, 111 bp, Artificial Sequence
  oligonucleotide for cloning ApoE ligand
gcggccgcgctgcgcaagctgcgtaagcggctcctccgcgatgccgatgacctgctgcgcaagctgcgtaagcggctcctccgcgatgccgatgacctgctcgagcaccat

SEQ ID NO: 23 - DNA, 111 bp, Artificial Sequence
  oligonucleotide for cloning ApoE ligand
atggtgctcgagcaggtcatcggcatcgcggaggagccgcttacgcagcttgcgcagcaggtcatcggcatcgcggaggagccgcttacgcagcttgcgcagcgcggccgc

SEQ ID NO: 24 - DNA, 42 bp, Artificial Sequence
  oligonucleotide for cloning a linker element
ggccccatcagcctccgcatctgcttccgcccctggatccgc

SEQ ID NO: 25 - DNA, 48 bp, Artificial Sequence
  oligonucleotide for cloning a linker element
ggccgcggatccaggggcggaagcagatgcggaggctgatggggccgc

SEQ ID NO: 26 - DNA, 1029 bp, Artificial Sequence
  Fusion protein comprising a sCAR, linker, trimerization domain and a ApoE ligand
atggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctctcgaaggaggaggaggaagtggaggaggaggaagtggaggaggaggaagtggatccgcggccatgaaacaaattgaagacaagattgaagaaattctatcaaaaatttatcacattgaaaacgaaattgccagaattaaaaaactaattggcgaagcggccccatcagcctccgcatctgcttccgcccctggatccgcggccgcgctgcgcaagctgcgtaagcggctcctccgcgatgccgatgacctgctgcgcaagctgcgtaagcggctcctccgcgatgccgatgacctgctcgagcaccatcaccatcatcac

SEQ ID NO: 27 - protein, 343 aa, Artificial Sequence
  Fusion protein comprising a sCAR, linker, trimerization domain and a ApoE ligand
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKALEGGGGSGGGGSGGGGSGSAAMKQIEDKIEEILSKIYHIENEIARIKKLIGEAAPSASASASAPGSAAALRKLRKRLLRDADDLLRKLRKRLLRDADDLLEHHHHHH

SEQ ID NO: 28 - DNA, 37 bp, Artificial Sequence
  Oligonucleotide for cloning the RGD ligand
ggccgcgtgcattgccgtggtgattgcttttgcgccc

SEQ ID NO: 29 - DNA, 38 bp, Artificial Sequence
  Oligonucleotide for cloning the RGD ligand
tcgagggcgcaaaagcaatcaccacggcaatcgcacgc

SEQ ID NO: 30 - DNA, 969 bp, Artificial Sequence
  Fusion protein comprising a sCAR, linker, trimerization domain and a RGD ligand
atggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctctcgaaggaggaggaggaagtggaggaggaggaagtggaggaggaggaagtggatccgcggccatgaaacaaattgaagacaagattgaagaaattctatcaaaaatttatcacattgaaaacgaaattgccagaattaaaaaactaattggcgaagcggccccatcagcctccgcatctgcttccgcccctggatccgcggccgcgtgcgattgccgtggtgattgcttttgcgccctcgagcaccatcaccatcatcac

SEQ ID NO: 31 - protein, 323 aa, Artificial Sequence
  Fusion protein comprising a sCAR, linker, trimerization domain and a RGD ligand
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKALEGGGGSGGGGSGGGGSGSAAMKQIEDKIEEILSKIYHIENEIARIKKLIGEAAPSASASASAPGSAAACDCRGDCFCALEHHHHHH

(19) United States
(12) Patent Application Publication
(10) Pub. No.: US 2002/0132769 A1
(43) Pub. Date: Sep. 19, 2002

(54) Targeting molecules

(76) Inventors: Michael Kaleko, Rockville, MD (US); Jin Gwan Kim, Germantown, MD (US); Theodore Smith, Ijamsville, MD (US); Susan C. Stevenson, Frederick, MD (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/971,798

(22) Filed: Oct. 5, 2001

Related U.S. Application Data
(60) Provisional application No. 60/327,562, filed on Oct. 6, 2000.

(51) Int. Cl. A61K 38/16; C07K 14/075

(52) U.S. Cl. 514/12; 530/350

(57) ABSTRACT

The present invention relates to targeting molecules which are useful to specifically target an adenoviral particle to a desired cell type. These targeting molecules comprise a soluble adenoviral receptor domain, a trimerization domain, and a targeting ligand domain. Further provided are polynucleotides encoding such targeting molecule, expression vectors including such polynucleotides, and methods to target an adenoviral particle to a cell, as well as methods to deliver a heterologous gene selectively to a cell.

This application claims the benefit under 35 USC §119(e) of the following United States provisional patent application: Provisional Application No. to be assigned, filed on Oct. 6, 2000, as application Ser. No. 09/684,552, for “Targeting Molecules.” The disclosure of that application is incorporated hereby by reference in its entirety.

### FIELD OF THE INVENTION

The present invention relates generally to the fields of molecular biology and gene therapy. More specifically, the present invention relates to the production of targeting molecules for the purpose of cell-specific targeting of adenoviral particles.

### BACKGROUND OF THE INVENTION

Adenoviruses are DNA viruses with genomes that are linear, double-stranded DNA molecules of about 36 kilobase pairs length. Each extremity of the viral genome has a short sequence known as the inverted terminal repeat (ITR), which is necessary for viral replication. The well-characterized molecular genetics of adenovirus render it an advantageous vector for gene transfer. The adenovirus delivers such genes to eukaryotic cells by binding to cellular receptors.

The adenovirus fiber protein is responsible for such attachment. The fiber protein consists of three domains: a tail domain, a rod-like shaft portion, and a globular head portion which contains the receptor binding region. The fiber spike is a homotrimer, and there are 12 spikes per virion. Human adenoviruses may bind to and infect a broad range of cultured cell lines and primary tissues from different species.

Adenovirus tropism is determined by attachment to specific cell surface molecules. Many adenovirus serotypes, except those in subgroup B, bind to a cell surface molecule called coxsackie-adenovirus receptor (CAR). Human CAR is a 365 amino acid transmembrane protein (46 kDa) with a short leader, a 222 amino acid extracellular domain, a membrane spanning helical domain, and a 107 amino acid intracellular domain (DNA sequence: Seq. Id. No.1; protein sequence: Seq. Id. No.2, see example 14). The extracellular region contains two immunoglobulin (Ig)-related structural domains. CAR is widely expressed in vivo, accounting for the widespread tissue distribution of systemically administered adenoviral vectors. Adenovirus serotype 3 (Ad3), which belongs to subgroup B, has been shown to bind to a different, as yet unidentified, receptor.

The ability of adenoviruses to infect a broad range of cell types, however, is not favorable if the desired target is a specific tissue or cell type. In addition to being inefficient, transduction of non-target cells may have undesirable effects. Successful vector targeting strategies may overcome these problems by directing the entire vector dose to the appropriate site. This may improve the safety profile of the vector and permit the use of lower vector doses, which would be less toxic and less immunogenic. Further, adenoviral vector targeting may permit transduction of cell types that are refractory to adenovirus infection, such as, for example, cells of the hematopoietic system or tumor cells. Thus, the targeting of adenoviral particles could expand the clinical utility of adenoviral vector-mediated gene therapy.

### SUMMARY OF THE INVENTION

The present invention provides novel targeting molecules which are useful to specifically target an adenoviral particle to a desired cell type. These targeting molecules comprise a soluble adenoviral receptor domain, a trimerization domain, and a targeting ligand domain. The trimerization domain facilitates the formation of trimeric versions of the targeting molecules, which are capable of forming stable complexes with adenoviral particles and demonstrate an enhanced ability to transduce target cells as compared to monomeric versions.

The targeting molecules of the invention ablate the natural tropism of adenoviral vectors and simultaneously redirect them to the target of choice. Furthermore, the targeting of adenoviral vectors with the described strategy does not require the time-consuming generation of modified adenoviral vectors. In contrast, adenoviral vectors that are employed in combination with the targeting molecules of the invention can be prepared and grown to high titer using normal protocols and standard cell lines. Thus, the described targeting molecules provide an efficient, rapid and facile means for purposes of cell-specific targeting.

Accordingly, the invention provides a method of targeting an adenoviral particle to a cell which expresses a cell surface molecule comprising the steps of contacting said adenoviral particle with the targeting molecule of the present invention to form a complex comprising said adenoviral particle and said targeting molecule and contacting said cell with said complex.

In the context of strategies to accomplish gene therapy, the invention further provides a method of delivering a heterologous gene selectively to a cell which expresses a cell surface molecule comprising the steps of 1) contacting an adenoviral particle which comprises said heterologous gene with the targeting molecule of the invention to form a complex comprising said adenoviral particle and said targeting molecule and 2) contacting said cell with said complex.

The flexibility of the targeting strategy of the invention also allows the rapid identification of cell surface molecules that are suitable to target with an adenoviral particle of the invention. Accordingly, the invention provides a method for identifying, either or both, a cell surface molecule that is suitable for mediating cell entry of an adenoviral particle to a specific cell or tissue, or a ligand that is suitable for targeting an adenoviral particle to a specific cell or tissue comprising the steps of 1) combining a ligand molecule for a cell surface molecule with a soluble adenoviral receptor molecule and a trimerization domain to form a targeting molecule, 2) contacting an adenoviral particle which comprises a marker gene with the targeting molecule to form a complex, 3) contacting a cell or tissue expressing said cell surface molecule with said complex, and 4) selecting a complex able to transduce efficiently said cell or tissue as reported by the marker gene.

Other and further aspects, features, and advantages of the present invention will be apparent from the following description of the presently preferred embodiments of the invention.

### BRIEF DESCRIPTION OF THE DRAWINGS

FIG. 1 shows schematic representations of soluble CAR constructs. The upper diagram represents the coxsackie-adenovirus receptor (CAR), including the leader sequence (LS), the extracellular domain, the transmembrane domain (TM) and the intracellular domain (ID). The 2nd diagram represents sCAR-cRGD which comprises the extracellular domain of CAR (sCAR) linked to a cRGD ligand. A His-tag was included at the carboxy-terminus to facilitate purification of the protein. The 3rd diagram represents sCAR-apoE which is similar to sCAR-cRGD except the apolipoproteinE (apoE) ligand replaces the cRGD ligand. The 4th diagram represents sCAR-trimer, which comprises a trimerization domain linked to the carboxy-terminus of the extracelluar domain of CAR (sCAR). The 5th diagram represents sCAR-trimer-apoE, which further comprises apoE as the targeting ligand domain.

FIG. 2 shows that the adenoviral vector-sCAR complex is stable. Human diploid fibroblasts (HDFs) were transduced with either unmodified Av3GFP (upper left) or Av3GFP complexed with sCAR-cRGD (upper right). Av3GFP-sCAR-cRGD complex was subjected to CsCl gradient centrifugation followed by freezing and thawing prior to transduction of HDFs (lower left). In addition to CsCl banding and freeze/thawing, the complex was exposed to serum for 60 minutes prior to transduction of HDFs (lower right). GFP fluorescence of the transduced cells shows that the complexes are stable under the tested conditions.

FIG. 3 shows that trimerized versions of sCAR containing a targeting ligand domain demonstrate an enhanced ability to transduce human diploid fibroblasts (HDFs) compared to monomeric versions of sCAR containing the same targeting ligand domain. Av3GFP without the addition of a sCAR fusion protein yields 8.3% GFP positive cells. sCAR fused with an apoE targeting ligand domain yields efficient transduction of HDFs.

FIG. 4 shows that sCAR-trimer effectively blocks liver transduction by the adenoviral vector. The monomeric version of sCAR does not block liver transduction efficiently. Group 1 mice did not receive any adenoviral vector. Group 2 mice received Av3nBg alone. Group 3 mice received Av3nBg complexed with 3 μg of sCAR trimer. Group 4 mice received Av3nBg complexed with 15 μg of sCAR trimer. Group 5 mice received Av3nBg complexed with 3 μg of sCAR monomer. Group 6 mice received Av3nBg complexed with 15 μg of sCAR monomer.

FIG. 5 shows the plasmid map of pcDNA3.1.

FIG. 6 shows the plasmid map of pClneo.

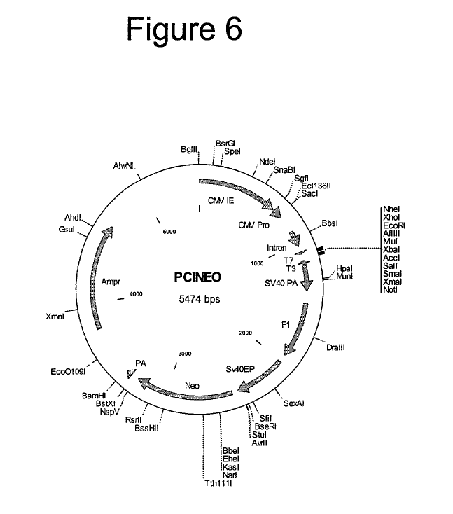

FIG. 7 shows that systemic delivery of oncolytic vectors using trimeric sCARt-cRGD targeting molecules reduces liver toxicity in tumor bearing mice. PC3 cell xenografts in nude mice were treated with a single intravenous administration of Ar6pAOscE3F alone (OV) or complexed with the trimeric sCARt-cRGD targeting molecule (OV+sCARt-cRGD) at 1×10¹³ particles per kilogram or with HBSS buffer alone. Blood was collected from 5 mice per group at 24 hours, 72 hours, or 1-week post injection and serum isolated and submitted for AST and ALT serum chemistries. The average AST (A) or ALT (B)±standard deviation is shown for each time point.

FIG. 8 shows that systemic delivery of oncolytic adenoviral vectors using trimeric sCARt-cRGD targeting molecules improves efficacy. PC3 cell xenografts in nude mice were treated with a single intravenous administration of Ar6pAOscE3F alone (OV) or complexed with the trimeric sCARt-cRGD targeting molecule (OV+sCARt-cRGD) at 1×10¹³ particles per kilogram or with HBSS buffer alone. Tumor volumes were determined twice a week. Results are shown as the average tumor volume (mm³)±standard error of the mean (SEM), (n=15 per group) over time. *, p=0.028, statistically significant difference compared with HBSS or virus alone treatment groups as determined by one way analysis of variance using the Tukey's test.

### DETAILED DESCRIPTION OF THE INVENTION

The present invention provides a targeting strategy that employs a soluble adenoviral receptor domain, such as the extracellular domain of CAR (sCAR). A targeting ligand domain is appended to the soluble adenoviral receptor domain, and then the conjugate is added to an adenoviral particle. The conjugate binds to the fiber knob of the adenoviral particle to form a complex and thereby redirects the vector to a different cell surface molecule. Surprisingly, it is found that trimerization of the soluble adenoviral receptor domain significantly enhances the binding of such a targeting molecule to the adenoviral particle. Furthermore, it is surprisingly found that adenoviral particles complexed with targeting molecules which include a trimerization domain and a targeting ligand domain more efficiently transduce cells in vitro and in vivo compared to targeting molecules without a trimerization domain. This approach of re-targeting an adenoviral particle does not require the time-consuming generation of genetically modified adenoviral vectors. Adenoviral particles can be prepared and grown to high titer using normal protocols and standard cell lines. The addition of a soluble adenoviral receptor domain, such as sCAR, fused to a targeting ligand domain ablates the normal tropism of the vector and simultaneously redirects it to the target of choice.

Accordingly, the present invention provides a targeting molecule comprising a soluble adenoviral receptor domain, a trimerization domain, and a targeting ligand domain.

An adenoviral receptor domain may bind an adenoviral fiber protein with an affinity of for example at least 1 μM (Kd) and preferably with an affinity of at least 1 nM (Kd). The adenoviral receptor domain masks the physiological binding epitope of the adenoviral fiber protein such that it can no longer interact with its physiological cellular receptor. Recent data describe the critical regions on the adenoviral fiber knob responsible for binding to CAR. These data show that the binding region is located on the side of the adenoviral fiber knob (Science 286 p.1568; Science 286 p.1579). An adenoviral receptor domain within the meaning of the invention may bind an adenoviral fiber protein of any one of the serotypes of human or nonhuman adenoviruses. Preferred, however, is the receptor for the Ad2 and the Ad5 adenovirus.

A soluble adenoviral receptor domain may be a fragment or a chemically modified fragment, or even the entire part of an adenoviral receptor molecule which retains binding specificity for an adenoviral fiber protein and may be dissolved in aqueous solution under physiological conditions. Preferably, the soluble adenoviral receptor domains are isolated extracellular domains of adenoviral receptor domains. In a preferred embodiment the soluble adenoviral receptor domain is sCAR. The CAR cDNA sequence is known in the art and is published under GenBank accession number Y07593. In one embodiment of the present invention sCAR comprises at least base pairs 60 to 487 of the published CAR cDNA sequence, extending from the ATG codon through the first Ig-like domain, termed the D1 domain. A preferred sCAR-sequence of this invention includes base pairs 54 to 767 of the CAR sequence.

The trimerization domain of the targeting molecule may be a heterologous trimerization domain with respect to the soluble adenoviral receptor domain, i.e. it comprises a nonnative amino acid sequence with respect to the soluble adenoviral receptor domain. “Nonnative amino acid sequence” encompasses any amino acid sequence that is not found in the soluble adenoviral receptor domain and which is introduced into the soluble adenoviral receptor domain, for example at the level of gene expression. Nonnative amino acid sequences include for example an amino acid sequence derived from a leucine zipper molecule, such as a yeast leucine zipper molecule. In one embodiment the nonnative amino acid sequence is a variant of the yeast leucine zipper molecule in which certain key leucine residues are mutated to isoleucine residues, such as in Harbury et al. Science 262 pp. 1401-1407 (1993). The trimerization domain confers upon the soluble adenoviral receptor domain the ability to form a trimer, in particular a homotrimer, directly or indirectly. Indirect homotrimerization may for example be achieved via a bispecific or multispecific binding agent, such as an antibody or fragment thereof, which interact with the trimerization domain.

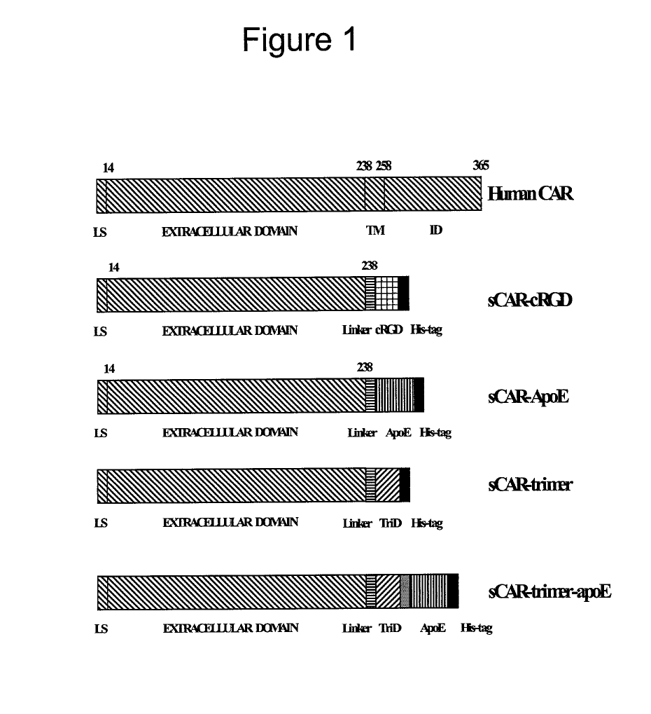

The trimerization domain may be localized downstream of the C-terminus of the soluble adenoviral receptor domain (FIG. 1, “sCAR-trimer” and “sCAR-trimer-apoE”). The trimerization domain may also be introduced into the sequence of the soluble adenoviral receptor domain. If the trimerization domain is introduced into the sequence of the soluble adenoviral receptor domain, it is preferably introduced into the carboxy-terminal end.

The trimerization domain of the invention may be derived in particular from the bacteriophage T4 fibritin molecule (Structure 5 p. 789, 1997), the neck region peptide from the human lung surfactant D protein (J Biol Chem 271 p.18912-18919, 1996 and J Immunol 164 p.5866-5870, 2000), a yeast heat shock transcription factor (Biochemistry 38 p.3559-3569, 1999), the trimerization domain of cartilage matrix protein (JMB 256 p. 909-923, 1996), the collagen-like tail of asymmetric acetylcholinesterase (Biochem J. 350 p.283-290, 2000), the trimer carboxyl propeptide of collagen I (J Biol Chem Aug. 2, 2000), the TNF_{α} trimerization domain (Microsc Res Tech 50 p.184-195, Aug 2000), the fusion protein of respiratory syncytial virus (J Virol 74 p.5911-5920, 2000) and procollagen (EMBO J 16 p.6694-6701, November 1997). In a particularly preferred embodiment the trimerization domain is the isoleucine variant of the yeast GCN4 protein (Seq. Id. No. 3: GCN4 variant DNA sequence: ATG AAA CAA ATT GAA GAC AAG ATT GAA GAA ATT CTA TCA AAA ATT TAT CAC ATT GAA AAC GAA ATT GCC AGA ATT AAA AAA CTA ATT GGC GAA; Seq. Id. No.4: GCN4 variant amino acid sequence: MKQIEDKIEEILSKIYHIENEIARIKKLIGE) as described in Harbury et al. Science 262 pp. 1401-1407 (1993), which is hereby incorporated by reference in its entirety.

It will be readily appreciated by the person skilled in the art that important criteria for selecting a suitable trimerization domain in a particular setting are, first, its “strength” and, second, its “size”. The strength of the trimerization domain may be quantified as the stability of the trimeric molecule formed under defined conditions, as measurable for example in its association/dissociation kinetics. The size of the trimerization domain (in particular the total number of amino acids of the trimerization domain) may be a criterion of choice in the construction of a particular targeting molecule because the trimerization domain should be small enough to be incorporated into the soluble adenoviral receptor domain without disrupting its binding function.

In yet another preferred embodiment the targeting molecule further comprises a linker element which is localized between the carboxy-terminal end of the adenoviral receptor domain and the trimerization domain (see FIG. 1). The linker element may preferably be a peptide linker. As used herein, the term “peptide linker” refers to a short peptide sequence serving as a spacer e.g. between the carboxy-terminal end of the adenoviral receptor domain and the trimerization domain. Such a sequence desirably is incorporated into the protein to ensure that the trimerization domains are not sterically hindered by the soluble adenoviral receptor domains and are capable to interact and efficiently form homotrimers. A linker sequence can be of any suitable length, preferably from about 3 to about 30 amino acids, and comprises any amino acids, for instance, a mixture of glycine and serine residues. Optimally, the linker sequence does not interfere with the functioning of the soluble adenoviral receptor domain. In a preferred embodiment the linker element consists of alternating glycine and serine residues.

The targeting molecule may also be assembled or combined, wholly or partly, by non-covalently binding each domain.

A targeting ligand domain of the targeting molecule of the present invention may include any cell specific attachment moiety which is suitable for the purposes of the invention. The targeting ligand domain may, for example, be selected from the group consisting of either physiological ligands, anti-receptor antibodies or cell specific peptides. The term “physiologic ligand” refers to a ligand for a cell surface receptor. Physiologic ligands and cell specific peptides derived therefrom are preferred elements of the targeting molecule of this invention.

A targeting ligand domain will selectively bind to a cell surface molecule. A targeting ligand domain “selectively binds” a cell surface molecule when it interacts with a binding domain of said cell surface molecule with a greater affinity, or is more specific for that binding domain as compared with other binding domains of other cell surface molecules. The expression “is specific for” refers to the degree of selectivity shown by a peptide or protein with respect to the number and types of interacting molecules with which the protein interacts and the rates and extent of these reactions, e.g. the degree of selectivity shown by an antibody with respect to the number and types of antigens with which the antibody combines and the rates and the extent of these reactions. The expression “selectively binds” in the present context also means binding sufficient to be useful in the method of the invention. As is known in the art, useful selective binding, for instance, to a receptor, depends on both the binding affinity and the concentration of ligand achievable in the vicinity of the receptor. Thus, binding affinities lower than that found for any naturally occurring competing ligands may be useful, as long as the cell or tissue to be treated can tolerate concentrations of added ligand sufficient to compete, for instance, for binding to a cell surface receptor.

The expression “cell surface molecule” within the meaning of the invention comprises any molecule displayed at the surface membrane of a eukaryotic cell which will selectively bind to a targeting ligand domain of the invention. By “cell surface molecule” is meant any site, i.e., a single molecule or a plurality of molecules, present on the surface of a cell with which the targeting ligand domain can interact to attach to the cell and, thereby, promote cell entry of the adenoviral particle.

A targeting ligand domain may be a fragment or a chemically modified fragment, or even the entire part of natural occurring ligand molecule specific to cell surface molecule(s).

Targeting ligands useful in the present invention include, but are not limited to, the TNF superfamily of ligands which include tumor necrosis factors (TNF's) such as, for example, TNF-alpha and TNF-beta, lymphotoxins, Fas ligand which binds to Fas antigen; CD40 ligand, which binds to the CD40 receptor of B-lymphocytes; CD30 ligand, which binds to the CD30 receptor of neoplastic cells of Hodgkin's lymphoma; CD27 ligand, NGF ligand, and OX-40 ligand; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-1 acid glycoprotein, which binds to the asialoglycoprotein receptor of liver; mannose-containing peptides, which bind to the mannose receptor of macrophages; sialyl-Lewis-X antigen-containing peptides, which bind to the ELAM-1 receptor of activated endothelial cells; CD34 ligand, which binds to the CD34 receptor of hematopoietic progenitor cells; ICAM-1, which binds to the LFA-1 (CD11b/CD18) receptor of lymphocytes, or to the Mac-1 (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-1 receptor of activated endothelial cells; LFA-1, which binds to the ICAM-1 receptor of activated endothelial cells; NGF, which binds to the NGF receptor of neural cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; interleukins 1 through 18 which bind to the interleukin 1 through 18 receptors, respectively; and the Fv antigen-binding domain of an immunoglobulin.

In one preferred embodiment the targeting ligand domain is chosen from FGF-2 (also known as basic fibroblasts growth factor-2) and EGF (epidermal growth factor). These targeting ligand domains are useful to target tumors.

In a further embodiment the targeting ligand domain is a single chain antibody fragment (scFv) targeting ligand domain. In particular the scFv may recognize cc39, which is a tumor specific marker. Accordingly this embodiment of the invention provides a further means to specifically target tumors.

In another preferred embodiment the targeting ligand domain is polylysine. Polylysine binds to heparan sulfate proteoglycans and is useful for locoregional delivery to tumors, i.e the choice of polylysine as the targeting ligand domain will enhance the infection of tumor cells throughout a tumor mass if the vector is locally injected into a tumor mass.

In a particular embodiment of this invention, the targeting ligand domain is cyclic RGD, which has the amino acid sequence CDCRGDCFC (Seq. Id. No.5 ). The cRGD ligand binds selectively to avβ3 and avβ5 integrins. Such integrins are found in abundance on proliferating endothelial cells, which are present in tumor blood vessels. It has been shown that cRGD homes to several tumor types, including carcinoma, sarcoma, and melanoma. Published data suggest that cRGD targets not only the tumor vasculature but also certain tumor cells themselves.

In a another preferred embodiment the targeting ligand domain includes at least 15 amino acids derived from an apoE protein. More preferred is a targeting molecule wherein the targeting ligand domain includes two tandem copies of amino acids 141-155 derived from an apoE protein (Seq. Id. No.6: apoE dimer DNA sequence: CTG CGC AAG CTG CGT AAG CGG CTC CTC CGC GAT GCC GAT GAC CTG CTG CGC AAG CTG CGT AAG CGG CTC CTC CGC GAT GCC GAT GAC CTG; Seq. Id. No.7: apo E dimer amino acid sequence: LRKLRKRLLRDADDL LRKLRKRLLRDADDL). The apoE ligand binds to receptors of the LDL-receptor family, which are found on hepatocytes, and, thus the ligand may be useful in liver gene therapy applications. Two tandem copies of the region of apolipoprotein E which recognizes the LDL receptor are used because it has been shown that this structure is required to generate an appropriate configuration for binding activity when the peptide is removed from its normal context.

In a further preferred embodiment of the present invention, the targeting ligand domain is conjugated to the carboxy-terminus of the soluble adenoviral receptor domain, either by covalent or non-covalent binding. Preferably, the targeting molecule further comprises a linker element which is localized between the carboxy-terminal end of the trimerization domain and the targeting ligand domain. Preferably, the linker inserted between the soluble adenoviral receptor domain and the trimerization domain is different in its amino acid sequence from the linker inserted between the trimerization domain and the targeting ligand domain in order to prevent homologies in the DNA construct encoding such a targeting molecule. Such homologies are disadvantageous because they may cause instability due to homologous recombination at the DNA level.

The present invention also provides a trimeric targeting molecule comprising the targeting molecule of the invention. The trimeric targeting molecule assembles due to the trimerization domain which has been introduced into the monomeric targeting molecule.

The present invention further provides a complex comprising an adenoviral particle and the targeting molecule. A “complex” of the adenoviral particle and the targeting molecule is any interaction, e.g., covalent or noncovalent, between the adenoviral particle and the targeting molecule. Preferably, it is a noncovalent interaction. Complex formation occurs when the adenoviral particle and the targeting molecule are contacted. Such “contacting” can be done by any means known to those skilled in the art and described herein, by which the mutual tangency of the adenovirus and targeting molecule can be effected. For instance, contacting of the adenoviral particle and the targeting molecule can be done by mixing these elements in a small volume of the same solution. For example, the adenoviral particle and the targeting molecule can be allowed to associate for 30 minutes at 37° C. in a suitable solution. Optionally, the adenoviral particle and the targeting molecule further can be covalently joined, e.g., by chemical means known to those skilled in the art, or other means, or, preferably, can be linked by means of noncovalent interactions (e.g., ionic bonds, hydrogen bonds, van der Waals forces, and/or nonpolar interactions). Preferably, the complex of the adenovirus and the targeting molecule is formed prior to the contacting of the cell. This period of time may be about as long as the maximum length of time a complex of an adenovirus and a targeting molecule can be stably maintained in a useable form, for instance, lyophilized, or in the presence of cryoprotective agents at −80° C.

The present invention also provides a polynucleotide encoding the amino acid sequence of the targeting molecule of the invention. Also provided is a polynucleotide that is a variant of such a polynucleotide and encodes a corresponding functional variant of the amino acid sequence of the targeting molecule. A functional variant may differ in amino acid sequence by one or more substitutions, additions, deletions, truncations which may be present in any combination, but would retain the same biological function as the referee targeting molecule.

Among preferred variants are those that vary from a reference polypeptide by conservative amino acid substitutions. Accordingly, the present invention also contemplates a polynucleotide encoding the amino acid sequence of the targeting molecule and any polynucleotide that is a conservatively modified variant of such a polynucleotide. A “conservatively modified variant” is a variation on the nucleic acid sequence that results in a conservative amino acid substitution. A “conservative amino acid substitution” is an amino acid substituted by an alternative amino acid of similar charge density, hydrophilicity/hydrophobicity, size, and/or configuration. In comparison, a “nonconservative amino acid substitution” is an amino acid substituted by an alternative amino acid of differing charge density, hydrophilicity/hydrophobicity, size, and/or configuration. The means of making such modifications are well known in the art.

The following non-limiting list of amino acids are considered conservative replacements: a) alanine, serine, and threonine; b) glutamic acid and asparatic acid; c) asparagine and glutamine d) arginine and lysine; e) isoleucine, leucine, methionine and valine and f) phenylalaine, tyrosine and tryptophan.

“Biological function” within the meaning of this application is to be understood in a broad sense. It includes, but is not limited to, the particular functions of the elements of the targeting molecule disclosed in this application, the element being the soluble adenoviral receptor domain, the trimerization domain and the targeting ligand domain. Thus, biological functions are not only those which a polypeptide displays in its physiological context, i.e. as part of a living organism or cell, but includes functions which it may perform in a non-physiological setting, e.g. in vitro. For example, a biological function of the soluble adenoviral receptor domain within the meaning of this application is the ability to bind to the fiber protein of an adenoviral particle of the invention either in vitro or in vivo. A biological function of the trimerization domain within the meaning of this application is the ability to trimerize the targeting molecule of the invention in vitro and to maintain the trimeric state in vivo. A biological function of the targeting ligand domain within the meaning of this application is the ability to bind to a corresponding cell surface molecule as defined in this application in vitro or in vivo. Assays to assess the required properties, for example the binding properties of the proteins to specific ligands are well-known in the art.

The means of making such a targeting molecule, in particular the means of introducing the sequence of the trimerization domain into the sequence of the soluble adenoviral receptor domain or at the 3′ end of the soluble adenoviral receptor domain at the level of DNA, is well known in the art, and is further described in the examples of the present invention. Briefly, the method comprises introducing a sequence of the chosen trimerization domain into the sequence encoding the chosen soluble adenoviral receptor domain so as to insert a new peptide motif into or in place of a protein sequence of the wild-type soluble adenoviral receptor domain. Such introduction can result in the insertion of a new peptide binding motif, or creation of a peptide motif, e.g. wherein some of the sequence comprising the motif is already present in the wild-type soluble adenoviral receptor domain. The method also can be carried out to replace sequences of the soluble adenoviral receptor domain with a nonnative amino acid sequence according to the invention. Generally, this can be accomplished by cloning the nucleic acid sequence encoding the soluble adenoviral receptor domain into a plasmid or some other vector for ease of manipulation of the sequence. Then, a unique restriction site at which further sequences can be added is identified or inserted into the sequence of the plasmid including the sequence of the soluble adenoviral receptor domain. A double-stranded synthetic oligonucleotide generally is created from overlapping synthetic single-stranded sense and antisense oligonucleotides such that the double-stranded oligonucleotide incorporates the restriction sites flanking the target sequence and, for instance, can be used to incorporate replacement DNA. The plasmid or other vector is cleaved with the restriction enzyme, and the oligonucleotide sequence having compatible cohesive ends is ligated into the plasmid or other vector to replace the wild-type DNA. Other means that are known to those skilled in the art, in particular using PCR techniques, can also be used to introduce the sequence of the trimerization domain into the soluble adenoviral receptor domain coding sequence.

In one preferred embodiment, the trimerization domain sequence is cloned so as to be located at the 3′ end of the sequence encoding the soluble adenoviral receptor domain. Further details of the cloning approaches provided by this invention are provided in the examples below.

The invention further provides an expression vector comprising a polynucleotide encoding the nucleic acid sequence of the targeting molecule, or comprising at least two polynucleotides encoding for a ligand molecule and a soluble adenoviral receptor molecule optionally further comprising in sequence a trimerization domain. A suitable expression vector is any vector that includes all necessary genetic elements for the expression of the inserted DNA sequence when propagated in a suitable host cell. Numerous suitable expression vectors are known to the person skilled in the art and are commercially available.

The present invention provides a complex comprising an adenoviral particle and the targeting molecule. The term “adenoviral particle” is to be understood broadly as meaning infectious viral particles that are formed when an adenoviral vector of the invention is transduced into an appropriate cell or cell line for the generation of infectious particles. The term “adenoviral vector” includes recombinant adenoviral vectors. Recombinant adenoviral vectors may be generated by a variety of techniques. Such techniques include introducing a desired gene of interest into a bacterial plasmid at a site flanked by adenovirus sequences. These sequences provide control elements for gene expression and serve as sites for recombination with a compatible adenoviral genome when cotransfected together into an appropriate mammalian cell line (Horwitz, M. S., “Adenoviruses, ” in Fields Virology, Third Edition, edited by B. N. Fields, D. M. Knipe, P. M. Howley et al., Lippincott-Raven Publishers: Philadelphia, Pa. (1996)).

The adenoviral vector which is employed may, in one embodiment, be an adenoviral vector which includes essentially the complete adenoviral genome. (Shenk, et al., Curr. Top. Microbiol. Immunol., (1984); 111(3):1-39). Alternatively, the adenoviral plasmid vector may be a modified adenoviral vector in which at least a portion of the adenoviral genome has been deleted.

In one embodiment, the vector comprises an adenoviral 5′ ITR; an adenoviral 3′ ITR; an adenoviral encapsidation signal; at least one DNA sequence encoding a therapeutic agent(s); and a promoter controlling the DNA sequence(s) encoding a therapeutic agent(s). The vector is free of at least the majority of adenoviral E1 and E3 DNA sequences, but is not free of all of the E2 and E4 DNA sequences, and DNA sequences encoding adenoviral proteins promoted by the adenoviral major late promoter. In one embodiment, the vector is also free of at least a portion of at least one DNA sequence selected from the group consisting of the E2 and E4 DNA sequences. In another embodiment, the vector is free of at least the majority of the adenoviral E1 and E3 DNA sequences, and is free of one of the E2 and E4 DNA sequences, and is free of a portion of the other of the E2 and E4 DNA sequences.

In yet another embodiment, the adenoviral vector is free of at least the majority of the E1 and E3 DNA sequences, is free of at least a portion of at least one DNA sequence selected from the group consisting of the E2 and E4 DNA sequences, and is free of DNA sequences encoding adenoviral proteins promoted by the adenoviral major late promoter.

Such an adenoviral vector may be constructed first by constructing, according to standard techniques, a shuttle plasmid which contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a tripartite leader sequence, a multiple cloning site (which may be as hereinabove described); a poly A signal; and a DNA segment which corresponds to a segment of the adenoviral genome. Such DNA segment serves as a substrate for homologous recombination with a modified or mutated adenovirus, and such sequence may encompass, for example, a segment of the adenovirus 5 genome no longer than from base 3329 to base 6246 of the genome. The plasmid may also include a selectable marker and an origin of replication. The origin of replication may be a bacterial origin of replication. A desired DNA sequence encoding a therapeutic agent may then be inserted into the multiple cloning site. Homologous recombination is then effected with a modified or mutated adenovirus in which at least the majority of the E1 and E3 adenoviral DNA sequences have been deleted. Such homologous recombination may be effected through co-transfection of the shuttle plasmid and the modified adenovirus into a helper cell line, such as 293 cells, by CaPO₄ precipitation. Upon such homologous recombination, a recombinant adenoviral vector is formed which includes DNA sequences derived from the shuttle plasmid between the Not I site and the homologous recombination fragment, and DNA derived from the E1 and E3 deleted adenovirus between the homologous recombination fragment and the 3′ ITR. The homologous recombination fragment preferably overlaps with nucleotides 3329 to 6246 of the adenovirus 5 genome.

Through such homologous recombination, an adenoviral vector may be formed which includes an adenoviral 5′ ITR, an adenoviral encapsidation signal; an E1a enhancer sequence; a promoter; a tripartite leader sequence; at least one DNA sequence encoding a therapeutic agent; a poly A signal; adenoviral DNA free of at least the majority of the E1 and E3 adenoviral DNA sequences; and an adenoviral 3′ ITR. This vector may then be transfected into a helper cell line, such as the 293 helper cell line, which will include the E1a and E1b DNA sequences, which are necessary for viral replication, and to generate infectious viral particles.

The adenoviral vector is transfected into an appropriate cell line for the generation of adenoviral particles. Transfection may take place by electroporation, calcium phosphate precipitation, microinjection, or through proteoliposomes. Examples of appropriate cell lines include, but are not limited to, HeLa cells or 293 (embryonic kidney epithelial) cells.

In another embodiment, the adenoviral vector comprises an adenoviral 5′ ITR; an adenoviral 3′ ITR; an adenoviral encapsidation signal; at least one DNA sequence encoding a therapeutic agent; and a promoter controlling the at least one DNA sequence encoding a therapeutic agent. The vector is free of the adenoviral E1 , E2, E3, and E4 DNA sequences, and the vector is free of DNA sequences encoding adenoviral proteins promoted by the adenoviral major late promoter.

Such vectors may be constructed by removing the adenoviral 5′ ITR, the adenoviral 3′ ITR, and the adenoviral encapsidation signal, from an adenoviral genome by standard techniques. Such components, as well as a promoter (which may be an adenoviral promoter or a non-adenoviral promoter), tripartite leader sequence, poly A signal, and selectable marker, may, by standard techniques, be ligated into a base plasmid or “starter” plasmid such as, for example, pBluescript II KS-(Stratagene), to form an appropriate cloning vector. The cloning vector may include a multiple cloning site to facilitate the insertion of DNA sequence(s) encoding therapeutic agent(s) into the cloning vector. In general, the multiple cloning site includes “rare” restriction enzyme sites; i.e., sites which are found in eukaryotic genes at a frequency of from about one in every 10,000 to about one in every 100,000 base pairs. An appropriate vector is thus formed by cutting the cloning vector by standard techniques at appropriate restriction sites in the multiple cloning site, and then ligating the DNA sequence encoding a therapeutic agent(s) into the cloning vector.

The vector may be packaged into infectious viral particles using a helper adenovirus which provides the necessary encapsidation materials. Preferably the helper virus has a defective encapsidation signal in order that the helper virus will not encapsidate itself. An example of an encapsidation defective helper virus which may be employed is described in Grable, et al., J. Virol., Vol. 66, pgs. 723-731 (1992).

The present invention also provides a method of targeting an adenoviral particle to a cell which expresses a cell surface molecule comprising the steps of contacting said adenoviral particle with a targeting molecule which comprises a soluble adenoviral receptor domain, a trimerization domain and a targeting ligand domain, obtaining a complex suitable to target said cell surface molecule and contacting said cell with said complex.

The present invention further provides a method of delivering an adenoviral vector selectively to a cell which expresses a cell surface molecule comprising the steps of contacting an adenoviral particle which comprises said adenoviral vector with a targeting molecule which comprises a soluble adenoviral receptor domain, a trimerization domain and a targeting ligand domain, obtaining a complex suitable to target said cell surface molecule, and contacting said cell with said complex.

Furthermore, the targeting molecules of the invention are useful to conveniently assess whether a cell surface molecule is capable of mediating cell entry of the adenoviral particle. One advantage of targeting vectors via sCAR is that it circumvents the need to generate modified vectors for each new target. In contrast, targeting strategies based on insertion of a targeting ligand domain into a capsid protein, such as fiber or hexon, or penton require the generation of a new adenoviral vector for each ligand to be tested. In addition, capsid protein modification strategies rely on a mechanism to ablate the normal tropism of the virus, such as generating mutations in the fiber knob to knockout CAR binding ability. However, ablation of CAR binding may adversely affect the entry of vector into packaging cells, requiring further manipulation of either the packaging cells or the virus to enable production of vector. The sCAR targeting strategy does not require manipulation of the virus or the packaging cells, so vectors can be grown to normal titers on standard packaging cells. In addition, the sCAR strategy simultaneously ablates the normal tropism of the vector and redirects it to a new target.

Thus, the present invention provides a convenient method to screen targeting ligand domains in cell culture. Numerous potential ligands can be inserted into the expression plasmid encoding sCAR and the corresponding fusion proteins can be generated conveniently. Each one can be complexed with an adenoviral vector encoding a marker gene, such as for example GFP, and the transduction efficiency of specific target cells can be rapidly assessed. Once the best ligands are identified, they may also be employed in other vector targeting strategies, such as the insertion of ligands into exposed loops of capsid proteins.

The present invention also provides a method of identifying a cell surface molecule that is suitable to mediate entry of an adenoviral particle to a specific cell or tissue expressing said cell surface molecule, comprising the steps of, producing a targeting molecule comprising a targeting ligand domain that interacts with said cell surface molecule, contacting an adenoviral particle which comprises a marker gene with the targeting molecule to form a complex, contacting said cell or tissue with said complex, and selecting a complex having efficiently transduced said cell or tissue as reported by the marker gene. Assessing the presence of said marker gene in said cell or tissue is sufficient for demonstrating efficient transduction. The “marker gene” may be any gene carried by the adenoviral vector, whether it is a part of adenoviral origin or heterologous, that can be monitored, e.g. by assessing the expression of the gene in a cell. Numerous useful methods to assess gene expression are known to the person skilled in the art.

Furthermore, the present invention also provides a method of delivering a heterologous gene selectively to a cell which expresses a cell surface molecule comprising the steps of contacting an adenoviral particle which comprises said heterologous gene with a targeting molecule which comprises a soluble adenoviral receptor domain, a trimerization domain and a targeting ligand domain, obtaining a complex which is suitable for targeting said cell surface molecule and contacting said cell with said complex.

Accordingly, the complexes of the invention may be administered in vivo to a host. The host may be an animal host, including mammalian hosts, primate hosts and human hosts. Thus, the complex of the invention is useful as a medicament and useful for the preparation of a medicament for the treatment of a disease in a mammal including a human.

The complex of the invention may be administered in an amount effective to provide a therapeutic effect in a host. In one embodiment, the vector may be administered in an amount of from 1 plaque forming unit to about 10¹⁴ plaque forming units, preferably from about 10⁶ plaque forming units to about 10¹³ plaque forming units. The host may be a human or non-human animal host. Preferably, the complex particles are administered systemically, such as, for example, by intravenous administration (such as, for example, portal vein injection or peripheral vein injection), intramuscular administration, intraperitoneal administration, or intranasal administration. The complex particles may be administered in combination with a pharmaceutically acceptable carrier suitable for administration to a patient. The carrier may be a liquid carrier (for example, a saline solution), or a solid carrier, such as, for example, microcarrier beads. The complex particles, travel directly to the desired cells or tissues upon the in vivo administration of such complex particles to a host. The targeted vector particles then infect the desired cell or tissues. Desired cells or tissues may, for example, be malignant cells and cancer tissue. Accordingly, the targeted vector particles are useful for the treatment of a disease such as for example cancer. For example, in one preferred embodiment the targeted vector particles are useful to treat prostate cancer such as, for example, adenocarcinoma of the prostate. Due to the re-targeting of the particles the side effects of the gene therapy may be reduced in such a method of treatment. In particular, the side effect of adenoviral liver toxicity may be reduced when complexes of the invention, rather than uncomplexed adenoviral vectors, are employed.

Cells which may be infected by the infectious complex particles include, but are not limited to, primary cells, such as primary nucleated blood cells, such as leukocytes, granulocytes, monocytes, macrophages, lymphocytes (including T-lymphocytes and B-lymphocytes), totipotent stem cells, and tumor infiltrating lymphocytes (TIL cells); bone marrow cells; endothelial cells; including activated endothelial cells; epithelial cells; keratinocytes; stem cells; hepatocytes, including hepatocyte precursor cells; fibroblasts; mesenchymal cells; mesothelial cells; parenchymal cells; vascular smooth muscle cells; brain cells and other neural cells; gut enterocytes; gut stem cells; and myoblasts. Cells which may be infected further include primary and metastatic cancer cells, including, but not limited to prostate, pancreatic, lung, including both small cell and non-small cell lung cancers, colon, and liver cancers. The cell which is “targeted” or infected or transduced with the infectious viral particles is dependent upon the ligand with which the targeting molecule has been engineered.

Accordingly, complexes of the invention are “targetable,” i.e., the complexes, upon administration to the host, will bind to and infect a desired target cell or tissue, and thereby deliver DNA encoding a therapeutic agent selectively to the desired target cell or tissue. The particular target cell or tissue to which the particles are targeted is dependent upon the ligand with which the targeting molecule is engineered, as the targeting ligand domain of the targeting molecule is specific for a cell surface molecule displayed by a desired target cell.

The present invention therefore also provides a method of targeting an adenovirus to a cell in order to accomplish cell entry of the adenoviral vector. The method comprises contacting the cell with a complex as described above such that entry of the adenovirus into the cell is effected. By “targeting” or “delivering selectively” it is meant to preferentially introduce into a particular cell rather than into another cell. According to the invention, a cell can be any cell, and, preferably, is a eukaryotic cell. Preferably, the eukaryotic cell is of a multicellular species, and, even more preferably, is a mammalian, e.g. human, cell. Desirably, such a eukaryotic cell is one in which an adenovirus can exist for a period of time (i.e., typically from anywhere up to, and potentially even after, about two months) after entry into the cell. Nascent RNA is transcribed from the adenoviral genome, which may include a nonnative gene, carried into the cell by the adenovirus, as further described herein.

A cell can be present as a single entity, or can be part of a larger collection of cells. Such a “larger collection of cells” can comprise, for instance, a cell culture (either mixed or pure), a tissue, e.g., epithelial or other tissue such as a neoplastic (benign or malign) tissue, an organ (e.g., heart, lung, liver and other organs), an organ system (e.g., circulatory system, respiratory system, gastrointestinal system, or other organ system), or an organism (e.g., a bird, mammal, or the like). In one embodiment, the cells being targeted are of the circulatory system (e.g., including, but not limited to heart, blood vessels, and blood), respiratory system (e.g., nose, pharynx, larynx, trachea, bronchi, bronchioles, lungs, and the like) or the gastrointestinal system (e.g., including mouth, pharynx, esophagus, stomach, intestines, salivary glands, pancreas, liver, gallbladder, and others). In a preferred embodiment cells of neoplastic tissue (i.e. ‘tumor tissue’) are targeted with the targeting molecule/adenoviral particle complex of the invention.

A cell to which a recombinant adenovirus is targeted differs from another cell, which is not targeted, in that the cell being targeted displays a particular cell surface molecule or at least a higher density of a particular cell surface molecule than a non-target cell. The cell surface molecule may be a protein (including a modified protein), a carbohydrate, a glycoprotein, a proteoglycan, a lipid, a mucin molecule or mucoprotein, and the like. Examples of potential cell surface molecules include, but are not limited to heparin and chondroitin sulfate moieties found on glycosaminoglycans; sialic acid moieties found on mucins, glycoproteins, and gangliosides; major histocompatability complex I glycoproteins; common carbohydrate molecules found in membrane glycoproteins, including mannose, N-acetyl-galactosamine, N-acetyl-glucosamine, fucose, and galactose; glycoproteins such as ICAM-1, VCAM, E-selectin, P-selectin, L-selectin, and integrin molecules; and tumor-specific antigens present on cancerous cells, such as, for instance, MUC-1 tumor-specific epitopes and the cc39 molecule. The targeting of tumor-specific antigens present on cancerous cells is one preferred embodiment of this invention. However, the present method of targeting an adenovirus to a cell is not limited to any specific mechanism of cellular interaction (i.e., interaction with a given cell surface binding site), and is not to be so construed.

The adenoviral particle complexed with the targeting molecules and including a gene encoding a desired protein or therapeutic agent may be employed to infect a desired cell line in vitro, whereby the infected cells produce a desired protein or therapeutic agent in vitro. The infected cells may be useful in the treatment of a variety of diseases (“ex vivo gene therapy”) including but not limited to adenosine deaminase deficiency, sickle cell anemia, thalassemia, hemophilia, diabetes, a-antitrypsin deficiency, brain disorders such as Alzheimer's disease, phenylketonuria and other illnesses such as growth disorders and heart diseases, for example, those caused by alterations in the way cholesterol is metabolized and defects of the immune system. The targeting molecules and complexes of the invention are also useful for the extracorporal transduction of blood vessels and organs, for example in the setting of transplantation applications of gene therapy, as well as in ex vivo cell therapy.

In a particularly preferred embodiment, oncolytic adenoviral particles are contemplated as the adenoviral particles of the invention. Oncolytic adenoviral particles are adenoviral particles which selectively replicate in tumor cells and destroy the cells in which they replicate, but do not replicate in non-tumor cells. They may or may not include a heterologous gene in addition to the adenoviral elements necessary for replication. In one embodiment oncolytic adenoviral particles include a mutation in a gene essential for adenoviral replication, such as the E1a or E1b genes. Such mutations may render adenoviral replication specific for tumor tissue, e.g. if the cells of said tissue have a defect in the p53 or Rb pathways. In another embodiment the oncolytic adenoviral particles may include a tissue-specific transcriptional regulatory sequence driving an adenoviral gene essential for replication of the adenoviral particle, which provides for tumor-tissue specific replication of the oncolytic adenoviral particles. See U.S. Pat. No. 5,998,205, issued Dec. 7, 1999 to Hallenbeck, et al.

In one aspect, the present invention provides a complex wherein the adenoviral particle includes a heterologous gene. The term “heterologous gene” means any gene that is not found in the corresponding naturally occurring (i.e. wild-type) adenovirus. The nonnative gene can be any gene, and desirably is either a therapeutic gene or a reporter gene, which, preferably, is capable of being expressed in a cell entered by the adenoviral particle. A therapeutic gene can be one that exerts its effect at the level of RNA or protein. For instance, a protein encoded by a therapeutic gene can be employed in the treatment of an inherited disease, e.g., the use of a cDNA encoding the cystic fibrosis transmembrane conductance regulator in the treatment of cystic fibrosis. Further, the protein encoded by the therapeutic gene can exert its therapeutic effect by causing cell death. For instance, expression of the protein, itself, can lead to cell death, as with expression of diphtheria toxin A, or the expression of the protein can render cells selectively sensitive to certain drugs, e.g., expression of the Herpes simplex thymidine kinase gene renders cells sensitive to antiviral compounds, such as acyclovir, gancyclovir and FIAU (1-(2-deoxy-2-fluoro-.beta.-D-arabinofuranosil)-5-iodouracil). Alternatively, the therapeutic gene can exert its effect at the level of RNA, for instance, by encoding an antisense message or ribozyme, a protein that affects splicing or 3′ processing (e.g. polyadenylation), or a protein that affects the level of expression of another gene within the cell, e.g. by mediating an altered rate of mRNA accumulation, an alteration of mRNA transport, and/or a change in post-transcriptional regulation. Thus, the use of the term “therapeutic gene” is intended to encompass these and any other embodiments of that which is more commonly referred to as gene therapy as known to those of skill in the art. The term “therapeutic agent” is used in a generic sense and includes treating agents, prophylactic agents, and replacement agents.

DNA sequences encoding therapeutic agents which may be placed into the adenoviral vector include, but are not limited to, DNA sequences encoding tumor necrosis factor genes, such as TNF-α; genes encoding interferons such as interferon-α, interferon-β, and interferon-γ; genes encoding interleukins such as IL-1, IL-1β, and interleukins 2 through 18; genes encoding GM-CSF; genes encoding adenosine deaminase, or ADA; genes which encode cellular growth factors, such as lymphokines, which are growth factors for lymphocytes; genes encoding soluble CD4; Factor VIII; Factor IX; T-cell receptors; the LDL receptor, ApoE, ApoC, ApoAl and other genes involved in cholesterol transport and metabolism; the alpha-1 antitrypsin gene, the ornithine transcarbamylase gene, the CFTR gene, the insulin gene, negative selective markers or “suicide” genes, such as viral thymidine kinase genes, such as the Herpes Simplex Virus thymidine kinase gene, the cytomegalovirus virus thymidine kinase gene, and the varicella-zoster virus thymidine kinase gene; Fc receptors for antigen-binding domains of antibodies, and antisense sequences which inhibit viral replication.

In a preferred embodiment of the invention the DNA sequence encoding the therapeutic agent is selected from either GM-CSF, thymidine kinase, Nos, FasL, or sFasR (soluble Fas receptor). The DNA sequence encoding the therapeutic agent may also be a sequence which is a part of the adenoviral genome, such as the adenoviral E1a gene. On one hand, E1a is instrumental in driving the adenoviral replication cycle, which in turn leads to cell lysis. Accordingly, E1a may be considered a DNA sequence encoding the therapeutic agent within the meaning of the invention, if administered to e.g. a tumor tissue. Furthermore, such genes may provide an additional therapeutical benefit, e.g. by sensitizing the infected cell to certain agents and/or radiation.

For human patients, the therapeutic gene will generally be of human origin although genes of closely related species that exhibit high homology and biologically identical or equivalent function in humans may be used if the gene does not produce an adverse immune reaction in the recipient. A therapeutic active amount of a nucleic acid sequence or a therapeutic gene is an amount effective at dosages and for a period of time necessary to achieve the desired result. This amount may vary according to various factors including but not limited to sex, age, weight of a subject, and the like.

The DNA sequence encoding at least one therapeutic agent is under the control of a suitable promoter. Suitable promoters which may be employed include, but are not limited to, adenoviral promoters, such as the adenoviral major late promoter; or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAl promoter. The following tumor selective promoters are preferred embodiments of this invention: E2F, osteocalcin, L-plastin, CEA, AVP, c-myc, telomerase, skp-2, psma, cyclin A, and cdc25 promoters. In a particularly preferred embodiment the promoter of the invention is the E2F promoter. In one embodiment of this invention the E2F promoter is operatively linked to the E1a gene. It is to be understood, however, that the scope of the present invention is not to be limited to specific foreign genes or promoters.

### EXAMPLES

The invention will now be described with respect to the following examples; it is to be understood, however, that the scope of the present invention is not intended to be limited thereby.

### Example 1

### Isolation of Human cDNA Encoding CAR

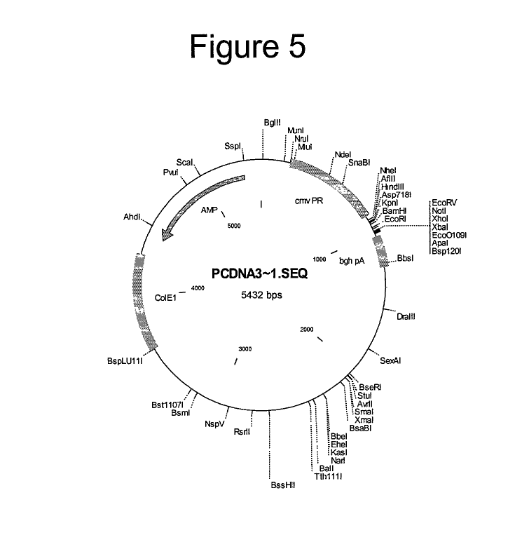

The human cDNA encoding CAR was cloned by RT-PCR. The published CAR cDNA sequence (GenBank accession number Y07593) was used to design oligonucleotide primers for amplification. Total RNA was isolated from cultured HeLa cells using RNAzol (Tel-Test, Friendswood, Tex.) and 1 μg was reverse transcribed using an RT-PCR system (Perkin Elmer). Primers were designed to amplify the entire coding sequence of the full-length CAR cDNA starting from the start codon, ATG, and ending with the termination codon, TAG. For cloning purposes, the sense primer contained a BamHI restriction site and a Kozak consensus sequence (5′-GTAGGATCCGCCACCATGGCGCTCCTGCTG-3′ (Seq. Id. No.8 )). The antisense primer contained an EcoRI restriction site (5′-GCGCGAATTCCTATACTATAGACCCATCCT-3′ (Seq. Id. No.9)). A 1.1 kb amplified product of the expected size was obtained and cloned into pcDNA3.1 (Invitrogen, San Diego, Calif.; FIG. 5) to generate the plasmid pcDNAhCAR. The nucleotide sequence of the cloned insert was determined and two individual basepair changes were identified in the intracellular domain. The first basepair change was silent and did not result in an amino acid change. The second basepair change resulted in a serine to asparagine amino acid change. The CAR extracellular domain was of the expected sequence.

### Example 2

### Construction of Recombinant Plasmids Encoding sCAR

To generate a plasmid expressing human sCAR with a histidine (his) tag at the carboxy-terminus, sequences encoding the extracellular domain of CAR were amplified by PCR from the pcDNAhCAR construct containing the entire CAR cDNA, described above. The sense primer contained an NheI site upstream of the ATG start codon and consisted of the sequence 5′-ACTAGCTAGCGCCGCCACCATGGCGCTC-3′ (Seq. Id. No. 10). The antisense primer encoded the end of the extracellular domain of CAR immediately followed by an XhoI site (to allow insertion of targeting ligand domains), a 6-his tag, a TAG stop codon, and an XbaI restriction site and consisted of the sequence 5′-GTGATCTAGACTAGTGATGATGGTGATGGTGCTCGAGAGCTTTATTTGAAGGAG GG-3′ (Seq. Id. No. 11). The PCR product was analysed by agarose gel electrophoresis, digested with NheI and XbaI, and ligated into the NheI and XbaI sites of the mammalian expression vector pCl-neo (Promega) to generate the plasmid pCl-neo-sCARa.

To permit insertion of targeting ligand domains at the end of sCAR, the following modifications were made to pCl-neo-sCARa. First, the NotI site in the multiple cloning region was destroyed by restriction digestion with NotI followed by blunting with T4 DNA polymerase and religating. Second, a pair of complementary oligonucleotides were synthesized and annealed to form a DNA duplex. The annealed oligonucleotides contained XhoI compatible overhangs at both ends and encoded a linker domain, to provide better presentation of the targeting ligand domain to its receptor, followed by a NotI restriction site, for insertion of ligands, and a factor Xa cleavage site, to permit removal of the his tag. The sequences of the oligonucleotides were as follows:

| | |
|---|---|
| 5′-TCGAACCATCAGCCTCCGCATCTGCTTCCGCCCCTGGATCCGCGGCCGCCATTG | (Seq. Id. No. 12) |
| AGGGCCGCC-3′ | |
| and | |
| 5′-TCGAGGCGGCCCTCAATGGCGGCCGCGGATCCAGGGGCGGAAGCAGATGCGG | (Seq. Id. No. 13) |
| AGGCTGATGGT-3′. | |

The annealed oligonucleotides were ligated into the XhoI site of pCl-neo-sCARa to generate the plasmid pCl-neo-sCARb.

To construct expression plasmids encoding targeting ligand domains at the carboxy-terminus of sCAR, pairs of complementary oligonucleotides were synthesized and annealed to form a DNA duplex encoding the desired targeting ligand domains. The DNA duplexes were designed to contain NotI compatible overhangs on both ends so the fragment could be inserted into the NotI site of pCl-neo-sCARb. Two different targeting ligand domains were fused to the end of sCAR. One ligand consists of the amino acid sequence CDCRGDCFC and is termed cyclic RGD (cRGD). The oligonucleotides that were synthesized to generate cRGD were as follows;

| | |
|---|---|
| 5′-GGCCTGCGATTGCCGTGGTGATTGCTTTTGCCC-3′ | (Seq. Id. No. 14) and |
| 5′-GGCCGCGCAAAAGCAATCACCACGGCAATCGCA-3′ | (Seq. Id. No. 15). |

The resulting plasmid was designated pCl-neo-sCAR-cRGD. The second targeting ligand domain consists of two tandem copies of amino acids 141-155 derived from apolipoproteinE (apoE) which binds to receptors of the LDL-receptor family. The oligonucleotides that were synthesized to generate the apoE targeting ligand domain were as follows:

| | |
|---|---|
| 5′-GGCCCTGCGCAAGCTGCGTAAGCGGCTCCTCCGCGATGCCGATGACCTGGC-3′ | (Seq. Id. No. 16) and |
| 5′-GGCCGCCAGGTCATCGGCATCGCGGAGGAGCCGCTTACGCAGCTTGCGCAG-3′ | (Seq. Id. No. 17). |

The resulting plasmid was designated pCl-neo-sCAR-apoE. A diagram of the structures encoded by pCl-neo-sCAR-cRGD and pCl-neo-sCAR-apoE is shown in FIG. 1.

Plasmids encoding trimerized sCAR were constructed as follows. First a pair of complementary oligonucleotides were synthesized and annealed to form a DNA duplex. The duplex contained XhoI compatible overhangs on each end and also encoded a glycine/serine linker element followed by a NotI site then a factor Xa cleavage site. The duplex was ligated into the XhoI site of pClneosCARa, described above, to generate the plasmid pClneosCARNot. The oligonucleotides were designed such that the 5′ XhoI site was destroyed, however, the 3′ site remained intact. The sequences of the complementary oligonucleotides were as follows:

| | |
|---|---|
| 5′ TCG AAG GAG GAG GAG GAA GTG GAG GAG GAG GAA GTG GAG GAG GAG | (Seq. Id. No. 18) |
| GAA GTG GAT CCG CGG CCG CCA TTG AGG GCC GCC 3′ | |
| and | |
| 5′ TCG AGG CGG CCC TCA ATG GCG GCC GCG GAT CCA CTT CCT CCT CCT | (Seq. Id. No. 19) |
| CCA CTT CCT CCT CCT CCA CTT CCT CCT CCT CCT 3′. | |

A second pair of complementary oligonucleotides were synthesized and annealed to form a DNA duplex encoding a trimerization domain derived from the isoleucine variant of the yeast GCN4 leucine zipper molecule. The duplex contained NotI compatible overhangs on both ends. The oligonucleotides were designed such that the 5′ NotI site was destroyed but the 3′ site remained intact. The sequences of the oligonucleotides were as follows:

| | |
|---|---|
| 5′ GGCC ATG AAA CAA ATT GAA GAC AAG ATT | (Seq. Id. No. 20) |
| GAA GAA ATT CTA TCA AAA ATT TAT CAC ATT GAA AAC GAA ATT GCC AGA | |
| ATT AAA AAA CTA ATT GGC GAA GC 3′ | |
| and | |
| 5′ GGCCGC TTC | (Seq. Id. No. 21) |
| GCC AAT TAG TTT TTT AAT TCT GGC AAT TTC GTT TTC AAT GTG ATA AAT | |
| TTT TGA TAG AAT TTC TTC AAT CTT GTC TTC AAT TTG TTT CAT 3′. | |

The duplex was ligated into the NotI site of pClneosCARNot to generate the plasmid pGStriCARa. Next, pGStriCARa was digested with both NotI and XhoI, the ends were blunted using Mung Bean Nuclease and ligated to generate the plasmid pGStriCARb, which encodes sCAR followed by a glycine/serine linker, the isoleucine variant GCN4 trimerization domain, and a 6 His Tag.

Plasmids encoding trimerized versions of sCAR containing an apoE targeting ligand domain were constructed as follows. First a pair of complementary oligonucleotides were synthesized and annealed to form a DNA duplex encoding the apoE targeting ligand domain consisting of two tandem copies of amino acids 141 to 155 derived from apolipoproteinE. The duplex contained a NotI site near the 5′ end and a XhoI site near the 3′ end. The sequences of the oligonucleotides were as follows:

| | |
|---|---|
| 5′ GCGGCC GCG CTG CGC AAG CTG CGT AAG CGG CTC CTC CGC GAT GCC | (Seq. Id. No. 22) |
| GAT GAC CTG CTG CGC AAG CTG CGT AAG CGG CTC CTC CGC GAT GCC | |
| GAT GAC CTG CTC GAG CAC CAT 3′ | |
| and | |
| 5′ ATG GTG CTC GAG | (Seq. Id. No. 23) |
| CAG GTC ATC GGC ATC GCG GAG GAG CCG CTT ACG CAG CTT GCG CAG | |
| CAG GTC ATC GGC ATC GCG GAG GAG CCG CTT ACG CAG CTT GCG CAG | |
| CGC GGC CGC 3′. | |

The duplex was digested with both NotI and XhoI and ligated into pGStriCARa which had been digested with both NotI and XhoI, to generate pGStriCARaapoE. Next, a linker element was inserted upstream of the targeting ligand domain as follows. A pair of complementary oligonucleotides were synthesized and annealed to generate a DNA duplex encoding a serine/alanine linker with NotI sites near each end. The sequences of the oligonucleotides were as follows:

| | |
|---|---|
| 5′ GGCC CCA TCA GCC TCC GCA TCT | (Seq. Id. No. 24) |
| GCT TCC GCC CCT GGA TCC GC 3′ | |
| and | |
| 5′ GGCC GC GGA TCC | (Seq. Id. No. 25) |
| AGG GGC GGA AGC AGA TGC GGA GGC TGA TGG GGC CGC 3′. | |

The duplex was digested with NotI and ligated into the NotI site of pGStriCARaapoE to generate pGStriCARapoE. The coding region of the sCAR-apoE fusion protein in this construct is shown in Seq. Id. No. 26.

A plasmid encoding a trimerized version of sCAR containing the cRGD targeting ligand was constructed as follows. First, a pair of complementary oligonucleotides was synthesized and annealed to form a DNA duplex encoding the cRGD ligand. The sequences of the oligonucleotides were as follows:

| | |
|---|---|
| 5′ GGCCGCGTGCATTGCCGTGGTGATTGCTTTTGCGCCC 3′ | (Seq. Id. No. 28) and |
| 5′ TCGAGGGCGCAAAAGCAATCACCACGGCAATCGCACGC 3′ | (Seq. Id. No. 29). |

At the 5′ end, the duplex contained an overhang compatible with a NotI site and the overhang on the 3′ end was compatible with a XhoI site. The duplex was ligated into the plasmid pGS-triCARapoE, which had been digested with both NotI and XhoI, to generate pGS-triCAR-RGD. The coding region of the sCAR-RGD fusion protein in this construct is shown in Seq. Id. No. 30.

### Example 3

### Production and Purification of sCAR Conjugated to a Targeting Ligand Domain

To generate sCAR protein conjugated to a targeting ligand domain, an expression plasmid encoding the fusion protein, either pCl-neo-sCAR-cRGD, pCl-neo-sCAR-apoE, pGStriCARb, or pGStriCARapoE was introduced into COS-7 cells by electroporation. Prior to electroporation the cells were resuspended in RPMI, 20% fetal bovine serum (FBS), HEPES at a final concentration of 1×10⁷ cells per milliliter. An aliquot of 500 μl of cells was placed into a 4 mm electroporation cuvette and 25 μg of plasmid DNA was added to the cells. Electroporation was performed using a BTX instrument with settings R3, 2100 μF, and 264 volts.

At various times after electroporation, the medium was removed from the cells and replaced with fresh medium. The medium was dialyzed against PBS to remove glutamine and any other components which may interfere with His-tag binding to the Ni-NTA resin (Qiagen). Ni-NTA resin was added to the medium and mixed for 2 hours at 4° C. The resin with bound protein was pelleted by centrifugation and washed several times with 50 mM NaH₂PO₄, 0.5 M NaCl, 60 mM imidazole, pH 8.0. Fusion proteins were eluted from the resin with 50 mM NaH₂PO₄, 0.5 M NaCl, 400 mM imidazole, pH 8.0. Removal of imidazole, buffer exchange, and concentration of purified proteins was performed using Centricon Plus-20 concentrators (Millipore). The purified proteins were stored at −70° C. in 1×PBS, 10% glycerol.

### Example 4

### Production and Purification of Trimerized sCAR Fused to an apoE Targeting Ligand Domain

To generate trimerized sCAR protein fused with an apoE targeting ligand domain, an expression plasmid encoding the fusion protein, pGStriCARapoE, was introduced into COS-7 cells by electroporation. The day after electroporation, the medium was removed from the cells and replaced with fresh medium. The next day, the cells were washed with PBS then lysed by adding 2 ml of RIPA buffer (5 mM Tris pH 8.0, 0.15 M NaCl, 1% deoxycholate, 1% NP-40) plus 1 mM PMSF (phenylmethylsulfonylfluoride). The cells were sheared by passing through a 21 gauge needle. The cell lysate was incubated on ice for 30 minutes. The lysate was centrifuged at 10,000 rpm for 20 minutes to remove cell debris. The supernatant was transferred to a clean tube and Ni-NTA resin was added (150 μl per 10 cm plate). The tube was rocked at 4° C. for 1 hour. The resin with bound sCAR fusion protein was collected by centrifugation at 3500 rpm for 5 minutes. The supernatant was removed and the resin was transferred to a clean tube and washed twice with 800 μl of Wash buffer (50 mM NaPO₄, 500 mM NaCl, 40 mM imidazole, 10% glycerol). The resin was washed a third time with Wash buffer containing 60 mM imidazole and a fourth time with Wash buffer containing 80 mM imidazole. The sCAR fusion protein was eluted from the resin using 100-400 μl Elution buffer (50 mM NaPO₄, 500 mM NaCl, 400 mM imidazole, 10% glycerol).

### Example 5

### Western Analysis of sCAR

The expression plasmid pClneosCAR, that encodes the extracellular domain of CAR with a His-Tag at the carboxy-terminus, was electroporated into COS-7 cells. Prior to electroporation the cells were resuspended at a concentration of 1×10⁷ cells per millimeter in RPMI medium containing 20% fetal bovine serum (FBS) and 10 mM HEPES (pH 7.4). An aliquot of 500 μl of cells was placed into a 4 mm electroporation cuvette and 25 μg of plasmid DNA was added to the cells. Electroporation was performed using a BTX instrument with settings R3, 2100 μF, and 264 volts. Media samples were collected on days 1, 2, 3, 4, and 7 after electroporation and the presence of sCAR was demonstrated by Western analysis using an anti-His-Tag antibody. The results showed that sCAR protein was continuously secreted from COS-7 cells for at least seven days. The identity of the protein was confirmed by a second Western analysis using purified lgG from rabbit antisera against sCAR as the probe.

### Example 6

### Generation of ³⁵S-labeled Ad5 Fiber and Cell Binding Assay

The ability of sCAR to bind adenovirus fiber protein was assessed by examining its ability to block binding of radioactively labeled fiber to cells. Ad5 fiber protein was produced and radioactively labeled in vitro using the T7 coupled transcription/translation reticulocyte lysate system (Promega). A 1 μg aliquot of plasmid DNA encoding fiber was incubated with the system components including 40 μCi of L-³⁵S-methionine in a total volume of 50 μl for 20 hours at 30° C. A 1 μl aliquot of the labeled protein mixture was analysed by non-denaturing SDS-4-15% PAGE and fluorography. The result showed significant levels of ³⁵S-labeled trimerized fiber protein. The ³⁵S-labeled fiber was mixed with various amounts of sCAR protein then applied to either HepG2 or HDF cell monolayers in a 6-well dish at a density of 1×10⁶ cells per well. ³⁵S-labeled cell bound protein were analysed by SDS-4-15% PAGE and fluorography. The results showed that fiber binding to cells was blocked by addition of increasing amounts of sCAR protein.

### Example 7

### Adenovirus-mediated Target Cell Transduction

Typically, sCAR protein was incubated with 2×10⁹ particles of Av3GFP, an E1 , E2a, E3 deleted adenoviral vector containing an RSV promoted green fluorescent protein (GFP) cDNA., for 30 minutes at room temperature in a total volume of 50 μl in PBS. Following incubation, the complex was diluted to 1 ml with the appropriate tissue culture medium plus 2% FBS, then added to 6-well plates containing 1×10⁶ cells, either human diploid fibroblasts (HDFs) or HepG2 cells. Cells were washed with 1×PBS prior to the addition of sCAR-adenoviral vector complex. Cells were incubated with the complex for 2 hours at 37° C. then the medium was changed and the cells were incubated at 37° C. another 24 to 48 hours. Cells were observed for green fluorescence under a Nikon fluorescence microscope. In some cases, to demonstrate specificity of binding, the cells were pre-blocked with either 10 μg per ml of competing Ad5 fiber protein or antibody against human LDL receptor or excess cRGD peptide for 30 minutes at 37° C. before addition of the sCAR-adenoviral vector complex. The results showed that trimerized sCAR protein, without a targeting ligand domain, efficiently blocked transduction of HepG2 cells. Monomeric sCAR protein also blocked transduction of HepG2 cells, although much greater amounts of monomeric sCAR protein were required to achieve the same degree of blocking.

### Example 8

### Complex Formation and Purification

A complex of Av3GFP vector with sCARcRGD protein was formed with a 550 times molar excess of sCAR to fiber monomer. Purified vector, 4×10¹¹ particles of Av3GFP in 200 μl, was mixed with 115 μl of partially purified sCARcRGD protein (400 μg) at room temperature for 30 minutes. The reaction mixture was then centrifuged in 1.33 g/ml CsCl at 60,000 rpm overnight in a Beckman Vti65.2 rotor. The virus-complex band was isolated and dialyzed into buffer containing 10% glycerol, 200 mM Tris, 50 mM HEPES adjusted to pH 8.0 with phosphoric acid. Particle concentration was determined by measuring absorbance at 260 nm.

### Example 9

### Targeting Adenovirus to Human Diploid Fibroblasts with sCAR-cRGD

The ability of sCAR conjugated to a targeting ligand domain to alter vector tropism was evaluated. First, pClneo-CAR-cRGD, was transfected into COS-7 cells by electroporation. Media was collected two, four, and six days after electroporation and sCAR-cRGD protein was isolated and partially purified by binding to Ni-NTA resin. The protein was mixed with an adenoviral vector encoding green fluorescent protein, Av3GFP, and the complex was added to a monolayer of human diploid fibroblasts (HDFs). Normally, HDFs are poorly transduced by adenovirus because the cells express little or no CAR on their surface. When unmodified Av3GFP was added to HDFs at a high multiplicity of infection (MOI), less than 1% of the cells expressed GFP. However, when sCAR-cRGD was added to Av3GFP, transduction of HDFs was efficient because the cells express the integrins recognized by cRGD.

### Example 10

### Targeting Adenovirus to HepG2 Cells with sCAR-ApoE

The receptors recognized by the apoE ligand are expressed at high levels on the surface of hepatocytes, including HepG2 cells which were derived from a hepatocellular carcinoma. Unmodified Av3GFP transduced HepG2 cells with high efficiency because hepatocytes express CAR on their surface. However, transduction was blocked in the presence of high levels of purified adenovirus fiber protein due to competition between purified fiber with fiber on the vector capsid for binding to CAR on the cell surface. Transduction of HepG2 cells with Av3GFP complexed with sCAR-apoE was not blocked by purified fiber protein, indicating that vector was bound to cells via the apoE ligand.

Further evidence that the apoE ligand was mediating the interaction with the cell via an LDL receptor was provided by competition experiments using an antibody against LDL receptor. Transduction of HepG2 cells by Av3GFP complexed with sCAR-apoE was blocked by anti-human LDL receptor antibody. In contrast, the antibody did not have any effect on transduction by unmodified Av3GFP.

### Example 11

### Stability of Soluble CAR Adenovirus Complex

To evaluate the stability of sCAR bound to an Av3GFP adenoviral vector, the ability to withstand centrifugation banding in cesium chloride followed by freezing, thawing and incubation in human serum for 60 minutes at room temperature was measured. A complex of Av3GFP with sCAR-cRGD was generated and CsCl banded as described in Example 8. The activity of CsCl banded complex was compared to the activity of freshly formed complex which was prepared by mixing 4×10⁹ particles of Av3GFP with 17.5 μg of sCAR-cRGD protein (2400 times molar excess of sCAR to fiber monomer) in a 50 μl reaction mixture in PBS for 30 minutes at room temperature. Human diploid fibroblasts (HDF) were cultured in 6-well dishes at 5×10⁵ cells per well. Cells were transduced at 37° C. in 2% FBS in DMEM media with 4×10⁹ particles per well of CsCl-banded complex. Control cells were transduced with 4×10⁹ particles per well of purified Av3GFP vector alone or freshly formed Av3GFP/sCARcRGD complex. Infection media was removed after two hours and replaced with 10% FBS in DMEM. Cells were monitored for GFP expression 24 hours later.

CsCl-banded Av3GFP/sCARcRGD complex was frozen in dialysis buffer and stored at −80° C. HDF cells were cultured in 6-well dishes at 1×10⁶ cells per well. Thawed complex, 4×10⁹ particles in 11 μl, was mixed with 89 μl of PBS and immediately put onto cells or incubated for 60 minutes in PBS at room temperature. Similarly, 4×10⁹ particles of thawed complex, 11 μl, was mixed with 89 μl normal mouse serum and incubated at room temperature for 30 minutes or 60 minutes. HDF cells were transduced with 4×10⁹ particles per well of thawed CsCl-banded complex with or without pre-incubation in normal mouse serum. Control cells were transduced with 4×10⁹ particles per well of purified Av3GFP vector alone or freshly formed Av3GFP/sCARcRGD complex for 2 hours at 37° C. in 2% FBS in DMEM media. Infection media was removed and replaced with 10% FBS in DMEM. Cells were monitored for GFP expression 24 hours later.

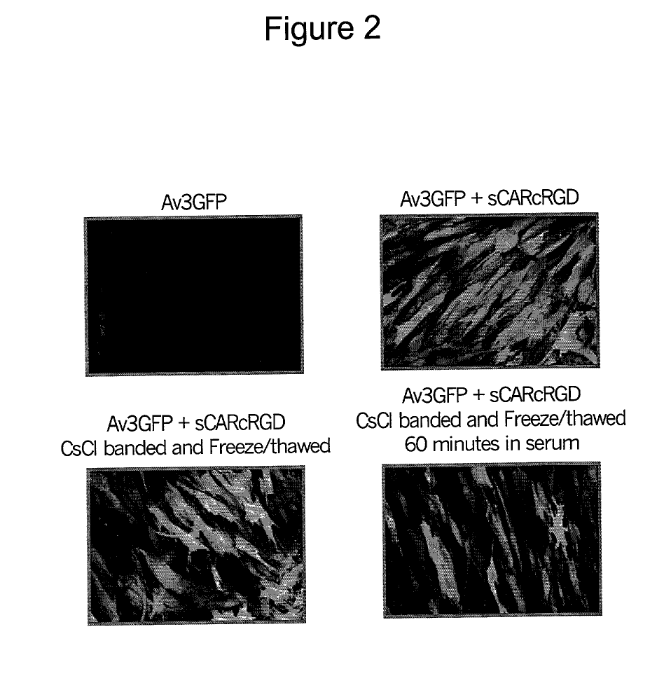

As shown in FIG. 2, unmodified Av3GFP did not transduce HDFs efficiently. This was expected because HDFs do not express CAR on their surface. However, Av3GFP complexed with sCARcRGD did transduce the cells efficiently. Additionally, the results showed that the Av3GFP/sCARcRGD complex was stable through CsCl banding, freezing and thawing, and exposure to serum.

### Example 12

### Trimerization of sCAR Significantly Enhances its Function

³⁵S-labeled Ad5 fiber protein was mixed with purified sCAR protein and subjected to electrophoresis on a non-denaturing acrylamide gel. Autoradiography of the dried gel demonstrated that ³⁵S-fiber was bound primarily to the trimeric form of sCAR. Bands were not visible at positions corresponding to monomeric or dimeric forms of sCAR. However, coomassie staining of non-denaturing gels showed that the large majority of sCAR was in a monomeric form. These observations suggested that trimers of sCAR had a significantly higher affinity for fiber than either monomers or dimers, but also suggested that trimers of sCAR were rare.

To increase the abundance of trimers of sCAR, a sequence encoding the GCN4 trimerization domain was inserted into the sCAR expression plasmid at the 3′ end of the sequence encoding the extracellular domain of CAR. To increase the likelihood that the trimerization domains would be accessible to one another to promote the formation of sCAR trimers, a linker consisting of alternating glycine and serine residues was inserted between the end of sCAR and the trimerization domain. The resulting expression plasmid was introduced into COS cells by electroporation and sCAR protein was isolated and purified as described above.

The ability of sCAR containing a trimerization domain to block transduction of HepG2 cells by the adenoviral vector Av3GFP was compared to that of sCAR without a trimerization domain. HepG2 cells were seeded into 6 well dishes at a density of 5×10⁵ cells per well. The next day, Av3GFP was treated with various amounts of both types of sCAR, either containing or lacking the trimerization domain. HepG2 cells were transduced with either untreated Av3GFP, Av3GFP treated with sCAR without a trimerization domain, or Av3GFP treated with sCAR containing a trimerization domain. Cells were transduced at an MOI of 3500 vector particles per cell. Cells were harvested 24 hours after transduction and GFP expression was measured by FACS analysis.

The results (Table 1) showed a significantly enhanced ability of trimerized sCAR to block transduction of HepG2 cells by Av3GFP. Experiment A shows the result for mock transduced cells, which was used to establish the division between GFP positive and negative cells. Cells transduced with unmodified Av3GFP were 98% positive (Experiment B). The extent to which sCAR without a trimerization domain was able to block transduction of cells is shown in Experiments C and D; 10 μg of sCAR reduced transduction to 79% positive cells and 20 μg of sCAR reduced transduction to 69% positive cells. The enhanced ability of trimerized sCAR to block transduction is demonstrated in Experiments E-G, where a significant shift of the peaks toward untransduced cells is observed.

TABLE 1

| Experiment | % GFP Positive |
|---|---|
| A Mock transduced | 0 |
| B Av3GFP | 98 |
| C Av3GFP + 10 μg monomeric sCAR | 79 |
| D Av3GFP + 20 μg monomeric sCAR | 69 |
| E Av3GFP + 5 μg trimeric sCAR | 43 |
| F Av3GFP + 10 μg trimeric sCAR | 32 |
| G Av3GFP + 20 μg trimeric sCAR | 32 |

Table 1. HepG2 cells were transduced with Av3GFP at an MOI of 3500 particles per cell. Twenty-four hours after transduction the cells were harvested and analyzed by FACS for GFP expression. (A) Mock transduced cells. (B) Cells transduced with is Av3GFP alone. (C) Cells transduced with Av3GFP in the presence of 10 μg of monomeric sCAR. (D) Cells transduced with Av3GFP in the presence of 20 μg of monomeric sCAR. (E) Cells transduced with Av3GFP in the presence of 5 μg of trimeric sCAR. (F) Cells transduced with Av3GFP in the presence of 10 μg of trimeric sCAR. (G) Cells transduced with Av3GFP in the presence of 20 μg of trimeric sCAR.

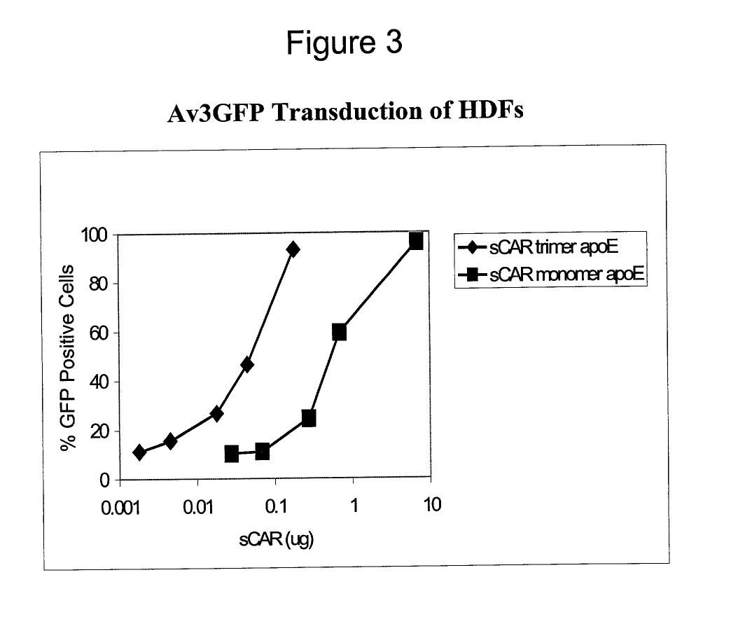

In addition, trimerized versions of sCAR containing a targeting ligand domain demonstrated an enhanced ability to transduce human diploid fibroblasts (HDFs) compared to monomeric versions of sCAR containing the same targeting ligand domain. The adenoviral vector Av3GFP was mixed with various amounts of either trimerized sCAR containing an apoE ligand or monomeric sCAR containing the apoE ligand. The adenoviral vector and sCAR fusion protein were allowed to associate for 30 minutes at 37° C. before adding the complex to HDFs, which had been seeded into the wells of a 6 well dish the prior day at a density of 5×10⁵ cells per well. In each case, an MOI of 5000 vector particles per cell was used. The day after transduction, the cells were harvested and analyzed by fluorescence activated cell sorting (FACS) for GFP expression. The results are shown in FIG. 3. HDFs express little or no CAR on their surface and therefore are poorly transduced by unmodified adenoviral vector. Av3GFP without the addition of a sCAR fusion protein yielded 8.3% GFP positive cells. As shown in FIG. 3, sCAR fused with an apoE targeting ligand domain yielded efficient transduction of HDFs. Notably, much lower amounts of the trimerized version of sCAR apoE were required to permit transduction of HDFs compared to the monomeric version. These results support the finding that trimerization of sCAR significantly enhances its function.

### Example 13

### Altered Tropism of an Adenoviral Vector in Vivo Using sCAR

This example demonstrates the utility of sCAR for targeting an adenoviral vector in vivo. The need for targeting is exemplified by the widespread tissue distribution of vector following peripheral vein administration. Preclinical studies in rodents, dogs, and non-human primates have demonstrated that an adenoviral vector transduces most organs and tissues examined following systemic administration. In mice, the highest level of transduction is seen in the liver, however, most other tissues show significant levels of vector. This example shows that trimerized sCAR without a targeting ligand domain can efficiently block transduction of the liver. In contrast, the monomeric form of sCAR does not effectively block liver transduction.

Av3nBg, an adenoviral vector encoding β-galactosidase, administered to C57BL/6 male mice via tail vein injection at a dose of 5×10¹⁰ particles per mouse. Cohorts of five mice received either unmodified adenoviral vector or vector complexed with either monomeric or trimeric sCAR. In cases where sCAR was added to the vector prior to injection, either 3 μg or 15 μg of sCAR protein was mixed with the adenoviral vector for 30 minutes at room temperature.

Three days after vector delivery, the animals were sacrificed and tissues, including liver, lung, heart, kidney, and spleen were collected. Slices of each tissue approximately 2-3 mm thick were placed in 10% neutral buffered formalin. After fixation, these samples were embedded in paraffin, sectioned, and analyzed by immunohistochemistry for β-galactosidase expression. Other pieces of tissue were placed in eppendorf tubes and frozen on dry ice. These tissue samples were used to isolate DNA for Southern analyses to determine the vector content and also to analyse β-galactosidase expression using a chemiluminescent assay (Tropix, Inc.). Standard procedures were used to isolate DNA from the tissues. Briefly, tissue is minced and digested overnight at 37° C. in 1% sodium dodecyl sulfate (SDS) and proteinase K (250 μg/ml). This is followed by phenol-chloroform extraction, Rnase treatment, a second phenol-chloroform extraction, chloroform-isoamyl alcohol extraction, ethanol precipitation, and resuspension in water. Ten micrograms of each DNA sample was digested with HindIII and subjected to Southern analysis. The probe was a radioactively labeled DNA fragment of the β-galactosidase cDNA.

The results showed expression of β-galactosidase in the liver following delivery of unmodified Av3nBg. In addition, Southern analysis showed that the liver contained significant levels of vector. Av3nBg that was mixed with monomeric sCAR prior to injection yielded the same levels of β-galactosidase expression in the liver and the same vector content in the liver as unmodified vector. However, Av3nBg complexed with trimerized sCAR showed significantly reduced β-galactosidase expression in the liver and reduced levels of vector in the liver, demonstrating that trimerized sCAR effectively blocks transduction.

Expression of β-galactosidase in the liver was assessed by immunohistochemical staining of tissue sections. In mice that received unmodified Av3nBg, approximately 10-15% of hepatocytes expressed β-galactosidase. Monomeric sCAR, had no effect on the percentage of hepatocytes expressing transgene;. In contrast, trimeric sCAR effectively blocked liver transduction so that less than 1% of hepatocytes were positive for β-galactosidase expression.

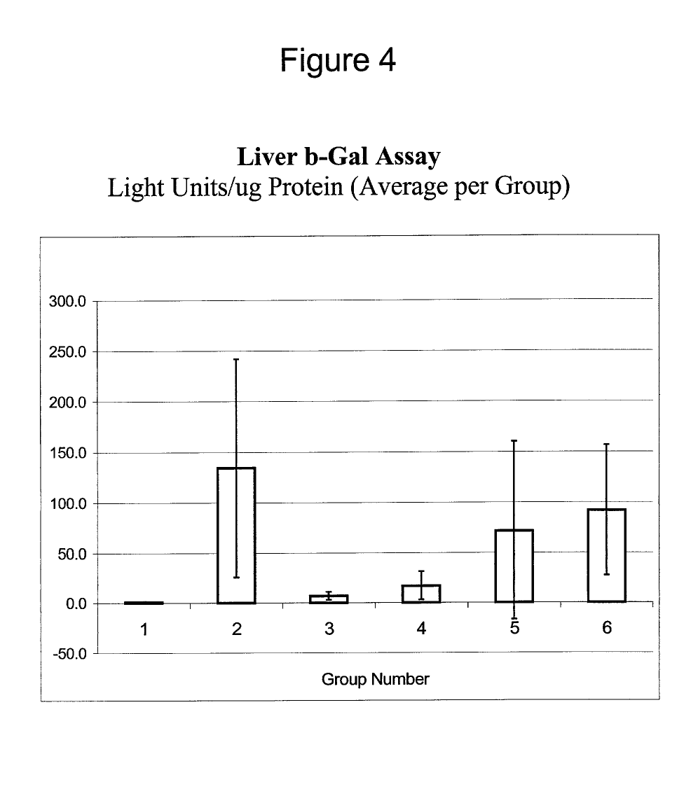

A second assay for β-galactosidase expression confirmed the results of immunohistochemical staining. The Galacto-Light Plus β-galactosidase reporter gene assay system (Tropix, Inc.) was used to measure β-galactosidase expression in liver samples. The results are shown in FIG. 4. Group 1 mice did not receive any adenoviral vector. Group 2 mice received Av3nBg alone. Group 3 mice received Av3nBg complexed with 3 μg of sCAR trimer. Group 4 mice received Av3nBg is complexed with 15 μg of sCAR trimer. Group 5 mice received Av3nBg complexed with 3 μg of sCAR monomer. Group 6 mice received Av3nBg complexed with 15 μg of sCAR monomer. The results showed that sCAR trimer effectively blocked liver transduction by the adenoviral vector. However, the monomeric version of sCAR did not block liver transduction efficiently. These results support the conclusion that trimerization of sCAR is required to achieve effective function.

### Example 14

### Human CAR Sequence

Seq. Id. No. 1: Human CAR DNA sequence (CDS =60-1157):

gaattcccag gagcgagagc cgcctacctg cagccgccgc ccacggcacg gcagccacca tggcgctcct
gctgtgcttc gtgctcctgt gcggagtagt ggatttcgcc agaagtttga gtatcactac tcctgaagag
atgattgaaa aagccaaagg ggaaactgcc tatctgccgt gcaaatttac gcttagtccc gaagaccagg
gaccgctgga catcgagtgg ctgatatcac cagctgataa tcagaaggtg gatcaagtga ttattttata
ttctggagac aaaatttatg atgactacta tccagatctg aaaggccgag tacattttac gagtaatgat ctcaaatctg
gtgatgcatc aataaatgta acgaatttac aactgtcaga tattggcaca tatcagtgca aagtgaaaaa
agctcctggt gttgcaaata agaagattca tctggtagtt cttgttaagc cttcaggtgc gagatgttac gttgatggat
ctgaagaaat tggaagtgac tttaagataa aatgtgaacc aaaagaaggt tcacttccat tacagtatga
gtggcaaaaa ttgtctgact cacagaaaat gcccacttca tggttagcag aaatgacttc atctgttata
tctgtaaaaa atgcctcttc tgagtactct gggacataca gctgtacagt cagaaacaga gtgggctctg
atcagtgcct gttgcgtcta aacgttgtcc ctccttcaaa taaagctgga ctaattgcag gagccattat aggaactttg
cttgctctag cgctcattgg tcttatcatc ttttgctgtc gtaaaaagcg cagagaagaa aaatatgaaa
aggaagttca tcacgatatc agggaagatg tgccacctcc aaagagccgt acgtccactg ccagaagcta
catcggcagt aatcattcat ccctggggtc catgtctcct tccaacatgg aaggatattc caagactcag
tataaccaag taccaagtga agactttgaa cgcactcctc agagtccgac tctcccacct gctaaggtag
ctgcccctaa tctaagtcga atgggtgcga ttcctgtgat gattccagca cagagcaagg atgggtctat
agtatagagc ctccatatgt ctcatctgtg ctctccgtgt tcctttcctt tttttgatat atgaaaacct attctggtct
aaattgtgtt actagcctca aaatacatca aaaaataagt taatcaggaa ctgtacggaa tatattttta aaaatttttg
tttggttata tcgaaatagt tacaggcact aaagttagta aagaaaagtt taccatctga aaaagctgga ttttctttaa
gaggttgatt ataaagtttt ctaaatttat cagtacctaa gtaagatgta gcgctttgaa tatgaaatca taggtgaaga
catgggtgaa cttacttgca taccaagttg atacttgaat aaccatctga aagtggtact tgatcatttt taccattatt
tttaggatgt gtatttcatt tatttatggc ccaccagtct cccccaaatt agtacagaaa tatccatgac aaaattactt
acgtatgttt gtacttggtt ttacagctcc tttgaaaact ctgtgtttgg aatatctcta aaaacataga aaacactaca
gtggtttaga aattactaat tttacttcta agtcattcat aaaccttgtc tatgaaatga cttcttaaat atttagttga
tagactgcta caggtaatag ggacttagca agctctttta tatgctaaag gagcatctat cagattaagt
tagaacattt gctgtcagcc acatattgag atgacactag gtgcaatagc agggatagat tttgttggtg
agtagtctca tgccttgaga tctgtggtgg tcttcaaaat ggtggccagc cagatcaagg atgtagtatc tcatagttcc
caggtgatat ttttcttatt agaaaaatat tataactcat ttgttgtttg acacttatag attgaaattt cctaatttat
tctaaatttt aagtggttct ttggttccag tgctttatgt tgttgttgtt tttggatggt gttacatatt atatgttcta
gaaacatgta atcctaaatt taccctcttg aatataatcc ctggatgata ttttttatca taaatgcaga ataatcaaat
acattttaag caagttaagt gtcctccatc aattctgtat tccagacttg ggaggatgta cagttgctgt tgtgtgatca
aacatgtctc tgtgtagttc cagcaaatca agctgagctt tgaaaaagtt tgtcttagtt ttgtgaaggt gatttattct
tagaaaaaaa aaaaaaaaaa aaaa

Seq. Id. No. 2: Human CAR amino acid sequence:

MALLLCFVLLCGVVDFARSLSITTPEEMIEKAKGETAYLPCKFT
LSPEDQGPLDIEWLISPADNQKVDQVIILYSGDKIYDDYYPDLKGRVHFTSNDLKSGD
ASINVTNLQLSDIGTYQCKVKKAPGVANKKIHLVVLVKPSGARCYVDGSEEIGSDFKI
KCEPKEGSLPLQYEWQKLSDSQKMPTSWLAEMTSSVISVKNASSEYSGTYSCTVRNR
V GSDQCLLRLNVVPPSNKAGLIAGAIIGTLLALALIGLIIFCCRKKRREEKYEKEVHHD
IREDVPPPKSRTSTARSYIGSNHSSLGSMSPSNMEGYSKTQYNQVPSEDFERTPQSPT
LPPAKVAAPNLSRMGAIPVMIPAQSKDGSIV

### Example 15

### Efficacy of the Oncolytic Adenovirus Ar6pAOscE3F Complexed with sCARt-cRGD: In Vivo Analysis of Trimeric sCAR Targeting Molecules in Tumor Bearing Mice

Methods

Adenoviral Vector. Ar6pAOscE3F is an oncolytic adenoviral vector containing the osteocalcin promoter to drive E1 expression and subsequent viral replication. A preparation of this vector was prepared using standard Cesium Chloride gradient purification methods.

Production of the trimeric sCAR targeting molecule. To generate the trimeric sCARt-cRGD targeting molecule used in these studies, the sCARt-cRGD expression plasmid was introduced into COS-7 cells by electroporation. The proteins were purified from cell lysates utilizing Ni-NTA resin (Qiagen) as described previously in Examples 3 and 4.

Cells. The human prostatic adenocarcinoma line PC3 (ATCC# CRL-1455) was obtained from American Type Culture Collection (Manassas, Va.). The PC3 cells were cultured in RPMI 1640 media containing 10% Fetal Bovine Serum.

Subcutaneous tumor xenograft model in nude mice. Female athymic outbred nu/nu mice (Harlen Sprague Dawley), 6 to 7 weeks of age, were implanted with 3×10⁶ PC3 cells subcutaneously in the right flank. Matrigel (Collaborative Biomedical Products) was added to the cells at a 1:1 ratio to enhance tumor formation. Tumor measurements were recorded (in two dimensions) twice-weekly using microcallipers. Tumor volumes were calculated using the formula Length×Width²×π/6. A study cohort of animals with tumor volumes of 90-200 mm³ were selected and randomly distributed into groups (n=20 per group, mean tumor volumes of 110 mm³) and injected intravenously with the virus alone or complexed with the trimeric sCAR targeting molecule as described in Table 2. A negative control group received the same volume of HBSS. The adenovirus and the trimeric sCAR targeting molecule were mixed at a 1:10 molar ratio and allow to interact for 30 minutes at room temperature prior to injection.

TABLE 2
Study design for the in vivo analysis of the trimeric
sCARt-cRGD targeting molecule

| | Mice | | Dose |
|---|---|---|---|
| Group | (n) | Test Article | (particles/kg) |
| 1 | 20 | HBSS | — |
| 2 | 20 | Ar6pAOscE3F | 1 × 10¹³ |
| 3 | 20 | Ar6pAOscE3F + | 1 × 10¹³ |
| | | sCARt-cRGD | |

On study days 1, 3, and 7 blood was collected from five animals per group for evaluation of liver toxicity. Tumor measurements were carried out twice per week for the duration of the study.

Blood was collected by retro-orbital sinus bleeds from 5 mice per group on study days 1, 3, and 7. Serum was collected and submitted to AniLytics, Inc. (Gaithersburg, Md.) for clinical chemistry analyses. Parameters analyzed were alanine aminotransferase (ALT) and aspartate aminotransferase (AST). Increased serum levels of both ALT and AST are indicative of hepatocellular injury.

Results

PC3 cells formed tumors of 90-200 mm³ approximately ten days after subcutaneous injection into nude mice. A single intravenous injection of virus alone or complexed with the trimeric sCAR targeting molecule at a dose of 1×10¹³ particles per kilogram was administered. At this viral dose there was no morbidity.

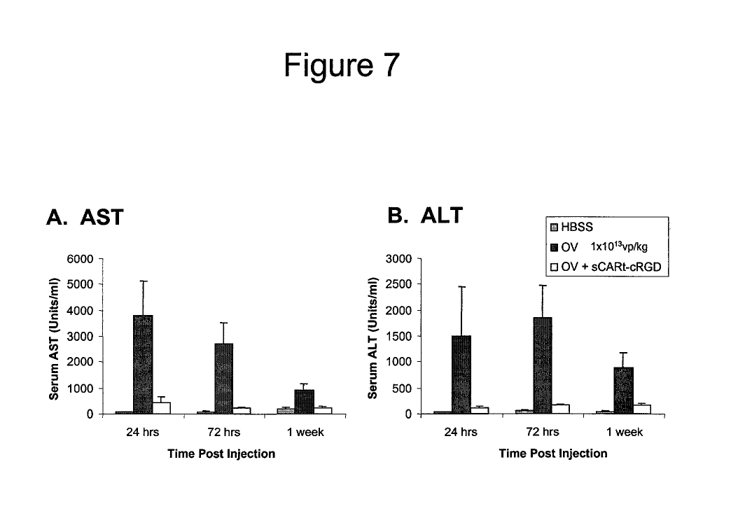

Blood samples were harvested from the mice on study days 1, 3, 7 and serum levels of ALT and AST were measured (FIG. 7). HBSS and Ar6pAOscE3F/sCARt-cRGD groups showed low levels of enzymes at all three time points. However the group treated with Ar6pAOscE3F showed elevated serum levels of ALT and AST compared to the other groups. Thus, adenoviral-mediated liver toxicity is reduced when oncolytic adenoviruses are systemically delivered using the trimeric sCARt-cRGD targeting molecule.

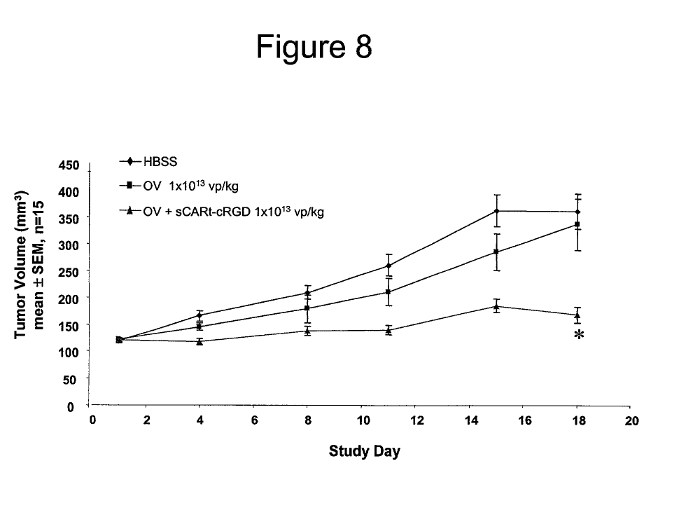

The ultimate goal of this study was to demonstrate that systemic delivery of oncolytic adenoviruses using trimeric sCARt targeting molecules to tumors leads to improved efficacy. To address this goal, the Ar6pAOscE3F oncolytic adenoviral vector was delivered intravenously alone or complexed with the sCARt-cRGD targeting molecule. Tumor volumes were determined twice per week for the duration of the study. FIG. 8 shows the average tumor volume over time for each treatment group. The average tumor volume for the HBSS and virus alone groups increased over time from a starting tumor volume of approximately 110 mm³ up to average tumor volumes of 359.8 and 335.4 mm³ at study day 18, respectively. When the Ar6pAOscE3F oncolytic adenovirus was delivered systemically using the sCARt-cRGD targeting molecule, the tumors did not increase in size as rapidly and resulted in a significantly lower tumor volume at study day 18 (p=0.028).

In summary, these in vivo results demonstrate that adenoviral vectors can be delivered systemically to specific sites using trimeric sCAR targeting molecules. Delivery of oncolytic adenoviral vectors using the sCARt-cRGD targeting molecule resulted in decreased liver transduction as shown by reduced liver toxicity and improved efficacy as shown by reduced tumor volumes.

The disclosures of all patents, publications (including published patent applications), and database accession numbers referred to in this specification are specifically incorporated herein by reference in their entirety to the same extent as if each such individual patent, publication and database number were specifically and individually indicated to be incorporated by reference in its entirety.

What is claimed is:

1. A targeting molecule comprising a soluble adenoviral receptor domain, a trimerization domain, and a targeting ligand domain.

2. The targeting molecule according to claim 1 wherein the soluble adenoviral receptor domain is sCAR.

3. The targeting molecule according to claim 1 wherein the trimerization domain is derived from a leucine zipper molecule.

4. The targeting molecule according to claim 1 wherein the trimerization domain is the isoleucine variant of the yeast GCN4 leucine zipper molecule.

5. The targeting molecule according to claim 1 wherein the trimerization domain is fused with the soluble adenoviral receptor domain.

6. The targeting molecule according to claim 5 wherein the trimerization domain is fused at the carboxy-terminal end of the soluble adenoviral receptor domain.

7. The targeting molecule according to claim 1 further comprising a linker element which is localized between the carboxy-terminal end of the soluble adenoviral receptor domain and the trimerization domain.

8. The targeting molecule according to claim 7 wherein the linker element consists of alternating glycine and serine residues.

9. The targeting molecule according to claim 1 wherein the targeting ligand domain is cyclic RGD.

10. The targeting molecule according to claim 1 wherein the targeting ligand domain includes at least 15 amino acids derived from an apoE protein.

11. The targeting molecule according to claim 10 wherein the targeting ligand domain includes two tandem copies of amino acids 141-155 derived from apoE protein.

12. The targeting molecule according to claim 1 wherein the targeting ligand domain is conjugated to the carboxy-terminus of the soluble adenoviral receptor domain.

13. The targeting molecule according to claim 1 further comprising a linker element which is localized between the carboxy-terminal end of the trimerization domain and the targeting ligand domain.

14. The targeting molecule according to claim 1 wherein the soluble adenoviral receptor domain is sCAR and the trimerization domain is derived from a leucine zipper molecule.

15. A trimeric targeting molecule comprising the targeting molecule according to claim 1.

16. A complex comprising an adenoviral particle and the targeting molecule according to claim 1.

17. The complex according to claim 16 wherein the soluble adenoviral receptor domain is sCAR.

18. The complex according to claim 16 wherein the trimerization domain is derived from a leucine zipper molecule.

19. The complex according to claim 16 further comprising a linker element which is localized between the carboxy-terminal end of the soluble adenoviral receptor domain and the trimerization domain.

20. The complex according to claim 16 wherein the soluble adenoviral receptor domain is sCAR and the trimerization domain is derived from a leucine zipper molecule.

21. The complex according to claim 16 wherein the adenoviral particle further comprises a heterologous gene.

22. The complex according to claim 16 wherein the adenoviral particle is an oncolytic adenoviral particle.

23. A polynucleotide encoding the targeting molecule according to claim 1.

24. An expression vector comprising a polynucleotide according to claim 23.

25. A method of targeting an adenoviral particle to a cell which expresses a cell surface molecule comprising the steps of (a) contacting an adenoviral particle with the targeting molecule of claim 1 to form a complex comprising said adenoviral particle and said targeting molecule and (b) contacting said cell with said complex.

26. The method of claim 25 wherein the adenoviral particle is an oncolytic adenoviral particle.

27. A method of delivering a heterologous gene selectively to a cell which expresses a cell surface molecule comprising the steps of (a) contacting an adenoviral particle which comprises said heterologous gene with the targeting molecule of claim 1 to form a complex suitable to target said cell surface molecule and (b) contacting said cell with said complex.

28. A method for identifying, either or both, a cell surface molecule that is suitable for mediating cell entry of an adenoviral particle to a specific cell or tissue expressing said cell surface molecule, or a ligand that is suitable for targeting an adenoviral particle to a specific cell or tissue, comprising the steps of, 1) combining a ligand molecule for a cell surface molecule with a soluble adenoviral receptor molecule and a trimerization domain to form a targeting molecule, 2) contacting an adenoviral particle which comprises a marker gene with the targeting molecule to form a complex, 3) contacting a cell or tissue expressing said cell surface molecule with said complex, and 4) selecting a complex able to transduce efficiently said cell or tissue as reported by the marker gene.

29. The targeting molecule according to claim 1, wherein the targeting ligand domain comprises a single chain antibody (scFv).

30. The complex of claim 16 for use as a medicament.

31. Use of the complex of claim 16 for the preparation of a medicament for the treatment of a disease in a mammal including a human.

32. The use of claim 31 wherein the disease is cancer.

33. The use of claim 32 wherein the cancer is an adenocarcinoma of the prostate.

34. A method for the treatment of a disease with adenoviral gene therapy comprising contacting a trimeric targeting molecule of claim 15 with an adenoviral gene therapy vector to form a complex, and administering said complex in a therapeutically effective amount to a patient in need thereof.

35. The method of claim 34 wherein the disease is cancer.

36. The method of claim 35, wherein said cancer is lung, colon, breast, prostate, or liver cancer.

37. The method of claim 35 wherein the cancer is an adenocarcinoma of the prostate.

38. The method of claim 34 wherein the side effects of the gene therapy are reduced.

39. The method of claim 34 wherein the side effect of adenoviral liver toxicity is reduced.
